(19) United States
(12) Patent Application Publication
(10) Pub. No.: US 2013/0171622 A1
(43) Pub. Date: Jul. 4, 2013

(54) COMPOSITIONS AND METHODS FOR DETECTING VIRAL INFECTION USING DIRECT-LABEL FLUORESCENCE IN SITU HYBRIDIZATION

(71) Applicant: Abbott Laboratories, Abbott Park, IL (US)

(72) Inventors: Ka-Cheung X. Luk, Lake Bluff, IL (US); John R. Hackett, JR., Libertyville, IL (US); Ekaterina Pestova, Abbott Park, IL (US)

(73) Assignee: Abbott Laboratories, Abbott Park, IL (US)

(21) Appl. No.: 13/728,019

(22) Filed: Dec. 27, 2012

Related U.S. Application Data
(60) Provisional application No. 61/581,919, filed on Dec. 30, 2011.

(51) Int. Cl. C12Q 1/70

(52) U.S. Cl. CPC C12Q 1/708
USPC 435/5

(57) ABSTRACT

The present invention relates generally to assays for the detection of viral infection and/or prognosis of viral infection and associated disease states. In particular, the invention relates to directly labeled viral-related nucleic acids having significant diagnostic, prognostic, and screening utilities and methods of using the same.

### CROSS REFERENCE TO RELATED APPLICATION

This application claims priority to U.S. Provisional Patent Application Ser. No. 61/581,919, filed on Dec. 30, 2011, which is incorporated by reference herein in its entirety.

### FIELD OF THE INVENTION

The present invention relates generally to assays for the detection of viral infection and/or prognosis of viral infection and associated disease states. In particular, the invention relates to directly-labeled viral-related nucleic acids having significant diagnostic, prognostic, and screening utilities and methods of using the same.

### BACKGROUND OF THE INVENTION

Fluorescence in situ hybridization (“FISH”) has been used extensively over the past 30 years for the detection of target nucleic acid sequences based on the ability of labeled probe sequences to specifically hybridize to, and thereby identify the presence of, such target nucleic acids. (Reviewed in Volpi et al., BioTechniques 45:385-409 (October 2008)). One factor that has facilitated the widespread expansion of FISH analysis into diverse areas of molecular biology, has been the development of new labels capable of signaling the hybridization of the probe to the target nucleic acid. These labels can be generally classified into three categories: (1) direct labels, where the probe itself is directly-labeled by a signal-generating moiety, e.g., by incorporation of a fluorescent nucleotide into the probe sequence; (2) indirect labels, where the probe includes a moiety that does not generate a signal itself, but which is subsequently detected by a signal-generating moiety, e.g., incorporation of a biotin-labeled nucleotide into the probe sequence that is bound by a fluorescently-labeled avidin molecule; and (3) labels capable of signal amplification, where the probe includes a moiety that may or may not generate its own signal, but which allows for the recruitment of a plurality of signal generating moieties in order to amplify the signal normally associated with hybridization of a probe to its target.

When applying FISH techniques to the detection of viral infection where the viral genome consists of less than about 10,000 base pairs (10 kb), methods employing indirect and/or amplified signal generation are overwhelmingly preferred over directly-labeled probes. For example, Morrison et al., note that “[f] or very small genomic targets, for example, targets less than 70 kilobases (kb), indirect labeling may be required to achieve visually interpretable staining. However, larger targets are usually detectable using direct labeling alone.” (Morrison et al., Methods in Molecular Biology, Vol. 204: Molecular Cytogenetics: Protocols and Applications, Ed. Y. S. Fan, Humana Press Inc., Totowa, N.J. (2003)). Viruses that fall within this range include, but are not limited to, Xenotropic Murine Leukemia Virus-related Virus (“XMRV”), Human T-cell Leukemia Virus (“HTLV”), Hepatitis C Virus (“HCV”), Human Immunodeficiency Virus (“HIV”), and Human Papillomavirus Virus (“HPV”). The art indicates that signal amplification is the preferred method of detecting such small viruses. (Adler et al., Histochem Cell Biol 108:321-324 (1997) (use of tyramide signal amplification to identify human papillomavirus); Plummer et al., Diag Mol Pathol 7:76-84 (1998) (use of catalyzed reporter deposition to increase the signal-generating potential of labeled hybridized probes to identify human papillomavirus); Evans et al., BMC Clin Pathol June 11; 3(1):2 (2003) (optimization of tyramide signal amplification techniques to identify human papillomavirus); Deichmann et al., J Virol Methods 65:19-25 (1997) (use of tyramide signal amplification to identify HIV-1); Matteucci et al., J Med Virol 74:473-483 (2004) (use of digoxigenin-labeled probe visualized by a fluorescence-based amplification system to identify HTLV-1); Januszkiewicz-Lewandowska et al., Jpn J Infect Dis 60:29-32 (2007) (use of biotin-labeled probe visualized by a fluorescence-based amplification system to identify HCV)).

The use of indirect and/or amplified signal generation when assaying for relatively short viral genomes is also considered preferable for the additional reason that, in certain instances, these viral genomes can be present in very low copy numbers, e.g., 1-10 copies per cell. For example, Montag et al. note that although indirectly-labeled FISH “offers detection and precise localization of DNA targets without destruction of morphology, it lacks high sensitivity, with a detection limit of 10-50 DNA copies/cell. Meanwhile, a highly sensitive ISH with signal amplification by tyramide has been developed allowing the detection of low-copy DNA.” (Montag et al., Arch Gynecol Obstet 284:999-1005 (2011) (internal citations omitted)).

The use of indirect and/or amplified signal generation is not without its drawbacks. For example, the use of an indirectly-labeled probe or an assay involving an amplified signal may require the use of cytology specimens as opposed to Formalin-Fixed Paraffin-Embedded (FFPE) tissue specimens. The difference between cytology specimen and FFPE tissue is that FFPE tissue contains inter-cellular materials in which cells of interest are embedded. Additionally, the tissue may contain residual cross-linked materials after pretreatment. In contrast, cell preparations (cytology specimens) contain isolated cells or groups of cells deposited onto the glass slide (the cell suspension is usually cleaned up prior to deposition to remove impurities by using washes or filters). Thus, the FFPE tissue can often be problematic in terms of potential nonspecific background signal caused by trapping of indirectly-labeled probe and/or amplified signal generating complexes. Additionally, signal amplification-based methods run the risk of amplifying any nonspecific background that may be present. Therefore, if sufficient sensitivity could be achieved, utilization of the direct-labeled probe, without intermediate steps of signal amplification, would be a preferred application for both FFPE and cytology specimens as the method yields low nonspecific background. Directly-labeled probe strategies have the additional advantages of requiring few sample handling steps, less costly components, and are less time-intensive.

The availability of a directly-labeled FISH assay capable of detecting viruses having genomes of less than about 10,000 base pairs would greatly facilitate diagnostic, prognostic, and screening methods for viral infection and viral infection-associated diseases.

### SUMMARY OF THE INVENTION

The present invention encompasses a method of detecting viral infection in a mammal comprising contacting a test sample obtained from the mammal with a directly-labeled nucleic acid composition capable of hybridizing to viral nucleic acids, wherein the presence of a signal indicative of hybridization to a viral nucleic acid sequence in the test sample indicates the presence of past or present viral infection in the mammal.

The present invention also provides methods for detecting viral nucleic acids that are indicative of viral infection, as well as disease states such as cancer, including, but not limited to: Acquired Immunodeficiency Syndrome (AIDS) and AIDS-related diseases, hepatocellular carcinoma, adult T-cell leukemia, hairy cell leukemia as well as cancers of the prostate, cervix, uterus, anus, oropharynx, penis, vagina, and vulva. In addition, the present invention provides methods for detecting viral nucleic acids that are indicative of a propensity to develop Acquired Immunodeficiency Syndrome (AIDS) and AIDS-related diseases, hepatocellular carcinoma, adult T-cell leukemia, hairy cell leukemia as well as cancers of the prostate, cervix, uterus, anus, oropharynx, penis, vagina, and vulva, and prognostic of the progression of these diseases.

The present invention also provides methods of the detection of viral RNA molecules within cells. In particular, viral RNA can be detected by directly-labeled FISH probes, including, but not limited to directly-labeled DNA FISH probes. In one such method, the DNA probe specific to the full-length viral genome is directly labeled with SO (SpectrumOrange: carboxytetramethylrhodamine, CTMR) fluorophore molecule, where this molecule is chemically attached to cytosine bases via an aminoethyl linker.

In certain methods, viral nucleic acids can be detected in cytology preparations as well as tissue specimens (sections of the formalin-fixed paraffin-embedded specimens). The patterns of viral nucleic acid detection observed in the certain experiments is “diffuse” FISH staining covering the cellular nucleus and extending into the cytoplasm, representing the nucleic acid migration from the nucleus into the cytoplasm. In certain methods, the patterns of viral nucleic acid detection observed in the certain experiments is “punctate” FISH staining in the cellular nucleus, the cytoplasm, or both.

The present invention also provides methods wherein RNA and DNA detection occur in the same cell. In particular, viral RNA and viral DNA can be detected by directly-labeled FISH probes. In one such method, a DNA probe specific to the full-length viral genome is directly labeled with SO fluorophore molecule, where this molecule is chemically attached to cytosine bases via an aminoethyl linker. Such methods provide opportunity of detecting total viral nucleic acids, and thus can be utilized for the detection of DNA and RNA viruses and their form (e.g., integrated provirus, episomal virus, actively replicating virus, low-level transcription of viral RNA). Additionally, the techniques described herein allow for detection of relatively small-length (e.g., approximately 5,000-10,000 base pairs) DNA fragments and RNA molecules.

The present invention also provides for diagnostic tests capable of detecting viral nucleic acids one or more sample types. For example, but not by way of limitation, a test can interrogate different tissue types including white blood cells, cytology specimens (e.g., urine, cervical brushing and swab specimens, esophageal brushing specimens, fine-needle aspirates, saliva, among others), and tissue specimens (such as archived FFPE tissue specimens).

### BRIEF DESCRIPTION OF THE FIGURES

FIG. 1A-B depict the results of XMRV FISH assays using a probe hybridization mix containing XMRV-SO probe (full-length XMRV VP62) and CEP8 internal control probe (complementary to a centromeric region of human chromosome 8) labeled with SA (SpectrumAqua, 7-diethylaminocoumarin-3-carboxylic acid, DECCA) fluorophore (CEP8-SA) in a mixture of human prostate cancer cell lines DU145 (uninfected; negative XMRV orange staining; three CEP8 aqua signals) and 22Rv1 (XMRV-infected; positive XMRV orange staining; two CEP8 aqua signals). (A) No RNase A treatment prior to hybridization. (B) With RNase A treatment prior to hybridization.

FIG. 2A-D depict the detection of XMRV nucleic acids in prostate cancer cell lines and tissue sections using FISH. Each slide was hybridized to a probe mix containing XMRV-SO viral probe and CEP8-SA internal control probe. No RNase A treatment was performed prior to hybridization. Representative images of XMRV-SO (orange) and the corresponding CEP8-SA (aqua) staining are shown in the left and right panels, respectively. (A) A mixture of prostate cancer cell lines DU145 (uninfected; negative XMRV orange staining; three CEP8 aqua signals) and 22Rv1 (XMRV-infected; positive XMRV orange staining, two CEP8 aqua signals). (B-D) Prostate cancer FFPE tissue sections (negative XMRV orange staining) of patients VP742, VP784 and VP800, respectively. Portions of the tissue sections with XMRV staining in the left panels are enlarged and illustrated in the corresponding right panels to display positive CEP8 aqua signals as denoted with arrows.

FIG. 3 depicts the results of HPV FISH assays using HPV16-SO probe in human cervical cancer cell line CaSki (positive control; carrying about 600 copies of integrated HPV-16 per cell), and in a mixture of human prostate cancer cell lines DU145 and 22Rv1 (negative controls). CEP8-SA from a centromeric region of human chromosome 8 is used as an internal control probe.

FIG. 4 depicts the results of an HPV FISH assay using HPV16-SO probe in human cervical cancer cell line SiHa (harboring 1 to 2 copies of integrated HPV-16 per cell). CEP8-SA from a centromeric region of human chromosome 8 is used as an internal control probe.

FIG. 5 depicts results of HPV FISH assays using HPV16-SO probe in FFPE human cervical cancer tissue specimen (V02-90, positive control) and human prostate cancer tissue specimen (26604, negative control). CEP8-SA from a centromeric region of human chromosome 8 is used as an internal control probe.

FIG. 6 depicts four more representative images of positive HPV orange staining observed in the FFPE tissue section of patient V02-90 probed with HPV16-SO as described in FIG. 5.

FIG. 7(A)-(C) depicts a comparison of a directly labeled probe and probe employing tyramide signal amplification for the detection of HPV-16 in tissue sections of head and neck squamous cell carcinoma (HNSCC). (A) HNSCC tissue section probed with biotinylated HPV-16 visualized with tyramide signal amplification (Fakhry et al., J Natl Cancer Inst 100:261-269 (2008)); (B)-(C) 11-09-X022B HNSCC tissue section probed with HPV16-SO and CEP8-SA.

FIG. 8 depicts an exemplary algorithm for HNSCC patient therapeutic stratification employing the techniques of the present application.

FIG. 9 depicts HPV16 Positive sample ID: M1120510A(Q), where two or many HPV16 signals per nucleus in many tumor cell nuclei are visible (HPV16 pos +++).

FIG. 10 depicts the status of HPV16-SO staining and two chromosomal FISH probes (MET and EGFR) on sixteen HNSCC specimens as outlined in Example 13.

FIG. 11 depicts the average ratios of MET/CEP7 and EGFR/CEP7 in each specimen tested as outlined in Example 13 (n=16).

FIG. 12 depicts the percentages of highly amplified and/or deleted cells were calculated for each of the two (MET and EGFR) and the ratio of MET/CEP7 and EGFR/CEP7, respectively, as outlined in Example 13.

FIG. 13 depicts the percent of HNSCC cases with various correlation patters of MET/EGFR as outlined in Example 13.

FIG. 14 depicts one example (sample ID: 04-06-A054A-1) of the combined HPV/FISH probe set containing HPV probe labeled with SO fluorophore, CEP 7 labeled with SA fluorophore, and EGFR labeled with SGn fluorophore (SpectrumGreen: 6-(fluorescein-5-(and-6)-carboxamido)hexanoic acid, FCHA) (HPV16-SO/EGFR-SGn/CEP7-SA) as employed in Example 13.

### DETAILED DESCRIPTION OF THE INVENTION

The present invention relates generally to assays for the detection of viral infection and/or prognosis of viral infection and associated disease states. Further, the subject invention relates to isolated and purified nucleic acid sequences or molecules which can be utilized in the detection and/or prognosis of viral infection and associated disease states. These utilities, as well as others, will be described, in detail, below. For purposes of clarity, and not by way of limitation, the detailed description is divided into the following subsections:
(i) definitions;
(ii) assay methods; and
(iii) diagnostic methods and kits.

### DEFINITIONS

For purposes of the present invention, “complementarity” is defined as the degree of relatedness between two DNA segments. It is determined by measuring the ability of the sense strand of one DNA segment to hybridize with the antisense strand of the other DNA segment, under appropriate conditions, to form a double helix. In the double helix, wherever adenine appears in one strand, thymine appears in the other strand. Similarly, wherever guanine is found in one strand, cytosine is found in the other. The greater the relatedness between the nucleotide sequences of two DNA segments, the greater the ability to form hybrid duplexes between the strands of two DNA segments.

A nucleic acid molecule is capable of “hybridizing” to another nucleic acid molecule when a single-stranded form of the nucleic acid molecule can anneal to the other nucleic acid molecule under the appropriate conditions of temperature and ionic strength (see Sambrook et al., “Molecular Cloning: A Laboratory Manual, Second Edition (1989), Cold Spring Harbor Laboratory Press, Cold Spring Harbor, N.Y.). The conditions of temperature and ionic strength determine the “stringency” of the hybridization. “Hybridization” requires that two nucleic acids contain complementary sequences. However, depending on the stringency of the hybridization, mismatches between bases can occur. The appropriate stringency for hybridizing nucleic acids depends on the length of the nucleic acids and the degree of complementation. Such variables are well known in the art. More specifically, the greater the degree of similarity, identity or homology between two nucleotide sequences, the greater the value of Tm for hybrids of nucleic acids having those sequences. For hybrids of greater than 100 nucleotides in length, equations for calculating Tm have been derived (see Sambrook et al., supra (1989)). For hybridization with shorter nucleic acids, the position of mismatches becomes more important, and the length of the oligonucleotide determines its specificity (see Sambrook et al., supra (1989)).

The term “identity” refers to the relatedness of two sequences on a nucleotide-by-nucleotide basis over a particular comparison window or segment. Thus, identity is defined as the degree of sameness, correspondence or equivalence between the same strands (either sense or antisense) of two DNA segments (or two amino acid sequences). “Percentage of sequence identity” is calculated by comparing two optimally aligned sequences over a particular region, determining the number of positions at which the identical base or amino acid occurs in both sequences in order to yield the number of matched positions, dividing the number of such positions by the total number of positions in the segment being compared and multiplying the result by 100. Optimal alignment of sequences can be conducted by the algorithm of Smith & Waterman, Appl. Math. 2:482 (1981), by the algorithm of Needleman & Wunsch, J. Mol. Biol. 48:443 (1970), by the method of Pearson & Lipman, Proc. Natl. Acad. Sci. (USA) 85:2444 (1988) and by computer programs which implement the relevant algorithms (e.g., Clustal Macaw Pileup (http://cmgm.stanford.edu/biochem218/11Multiple.pdf; Higgins et al., CABIOS. 5L151-153 (1989)), FASTDB (Intelligenetics), BLAST (National Center for Biomedical Information; Altschul et al., Nucleic Acids Research 25:3389-3402 (1997)), PILEUP (Genetics Computer Group, Madison, Wis.) or GAP, BESTFIT, FASTA and TFASTA (Wisconsin Genetics Software Package Release 7.0, Genetics Computer Group, Madison, Wis.). (See U.S. Pat. No. 5,912,120.)

As used herein, an “isolated nucleic acid fragment or sequence” is a polymer of RNA or DNA that is single or double-stranded, optionally containing synthetic, non-natural or altered nucleotide bases. An isolated nucleic acid fragment in the form of a polymer of DNA can be comprised of one or more segments of cDNA, genomic DNA or synthetic DNA. (A “fragment” of a specified polynucleotide refers to a polynucleotide sequence which comprises a contiguous sequence of approximately at least about 6 nucleotides, at least about 8 nucleotides, at least about 10 nucleotides, at least about 15 nucleotides, at least about 25 nucleotides, and can be up to the full length of the reference sequence, up to the full length sequence minus one nucleotide, or up to 50 nucleotides, 100 nucleotides, 500 nucleotides, 1000 nucleotides, 2000 nucleotides, 3000 nucleotides, 4000 nucleotides, 5000 nucleotides, 6000 nucleotides, 7000 nucleotides, 8000 nucleotides, 9000 nucleotides, or 10,000 nucleotides identical or complementary to a region of the specified nucleotide sequence.) Nucleotides (usually found in their 5′ monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The terms “fragment or sub-fragment that is functionally equivalent” and “functionally equivalent fragment or sub-fragment” are used interchangeably herein. These terms refer to a portion or subsequence of an isolated nucleic acid fragment in which the ability to alter gene expression or produce a certain phenotype is retained whether or not the fragment or sub-fragment encodes an active enzyme. For example, the fragment or sub-fragment can be used in the design of chimeric constructs to produce the desired phenotype in a transformed plant. Chimeric constructs can be designed for use in co-suppression or antisense by linking a nucleic acid fragment or sub-fragment thereof, whether or not it encodes an active protein, in the appropriate orientation relative to a promoter sequence.

The terms “homology”, “homologous”, “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the present invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences described herein.

“Gene” refers to a nucleic acid fragment that expresses a specific protein, including regulatory sequences preceding (5′ non-coding sequences) and following (3′ non-coding sequences) the coding sequence.

“Native gene” refers to a gene as found in nature with its own regulatory sequences. In contrast, “chimeric construct” refers to a combination of nucleic acid fragments that are not normally found together in nature. Accordingly, a chimeric construct can comprise regulatory sequences and coding sequences that are derived from different sources, or regulatory sequences and coding sequences derived from the same source, but arranged in a manner different than that normally found in nature. (The term “isolated” means that the sequence is removed from its natural environment.)

A “foreign” gene refers to a gene not normally found in the host organism, but that is introduced into the host organism by gene transfer. Foreign genes can comprise native genes inserted into a non-native organism, or chimeric constructs. A “transgene” is a gene that has been introduced into the genome by a transformation procedure.

In certain embodiments, such as, but not limited to, in situ hybridization assays, such as FISH assays, a “probe” or “primer” as used herein refers to a polynucleotide that is at least 10 nucleotides, at least 100 nucleotides, at least 1000 nucleotides, at least 2000 nucleotides, at least 3000 nucleotides, at least 4000 nucleotides, at least 5000 nucleotides, at least 6000 nucleotides, at least 7000 nucleotides, or at least 8000 nucleotides, and in certain embodiments can refer to a polynucleotide that is up to up to 1,500,000 nucleotides.

“Coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences can include, but are not limited to, promoters, translation leader sequences, introns, and polyadenylation recognition sequences.

“RNA transcript” refers to the product resulting from RNA polymerase-catalyzed transcription of a DNA sequence. When the RNA transcript is a perfect complementary copy of the DNA sequence, it is referred to as the primary transcript or it can be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a DNA that is complementary to and synthesized from a mRNA template using the enzyme reverse transcriptase. The cDNA can be single-stranded or converted into the double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA and that blocks the expression of a target gene (U.S. Pat. No. 5,107,065). The complementarity of an antisense RNA can be with any part of the specific gene transcript, i.e., at the 5′ non-coding sequence, 3′ non-coding sequence, introns, or the coding sequence. “Functional RNA” refers to antisense RNA, ribozyme RNA, or other RNA that can not be translated but yet has an effect on cellular processes. The terms “complement” and “reverse complement” are used interchangeably herein with respect to mRNA transcripts, and are meant to define the antisense RNA of the message.

The term “endogenous RNA” refers to any RNA which is encoded by any nucleic acid sequence present in the genome of the host prior to infection by a virus, whether naturally-occurring or non-naturally occurring, i.e., introduced by recombinant means, mutagenesis, etc.

The term “non-naturally occurring” means artificial, not consistent with what is normally found in nature.

The term “operably linked” refers to the association of two moieties. For example, but not by way of limitation, the association of two or more nucleic acid sequences on a single nucleic acid fragment so that the function of one is regulated by the other. In one such non-limiting example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another non-limiting example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA. Alternative examples of operable linkage include, but are not limited to covalent and noncovalent associations, e.g., the biotinylation of a polypeptide (a covalent linkage) and hybridization of two complementary nucleic acids (a non-covalent linkage).

The term “expression”, as used herein, refers to the production of a functional end-product. Expression of a gene involves transcription of the gene and translation of the mRNA into a precursor or mature protein. “Antisense inhibition” refers to the production of antisense RNA transcripts capable of suppressing the expression of the target protein. “Co suppression” refers to the production of sense RNA transcripts capable of suppressing the expression of identical or substantially similar foreign or endogenous genes (U.S. Pat. No. 5,231,020).

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

The term “recombinant” refers to an artificial combination of two otherwise separated segments of sequence, e.g., by chemical synthesis or by the manipulation of isolated segments of nucleic acids by genetic engineering techniques.

The terms “recombinant construct”, “expression construct” and “recombinant expression construct” are used interchangeably herein. These terms refer to a functional unit of genetic material that can be inserted into the genome of a cell using standard methodology well known to one skilled in the art. Such a construct can be itself or can be used in conjunction with a vector. If a vector is used, then the choice of vector is dependent upon the method that will be used to transform host plants, as is well known to those skilled in the art. For example, a plasmid can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., EMBO J 4:2411-2418 (1985); De Almeida et al., Mol Gen Genetics 218:78-86 (1989)), and thus that multiple events must be screened in order to obtain lines displaying the desired expression level and pattern. Such screening can be accomplished by Southern analysis of DNA, Northern analysis of mRNA expression, Western analysis of protein expression, or phenotypic analysis.

### Assay Methods

Assays Employing Directly-Labeled Probes

In certain embodiments, the present invention provides compositions and methods for the detection of viral nucleic acids using nucleic acid hybridization-based assays. In certain embodiments, the methods for detection via hybridization of the present invention include, but are not limited to fluorescence in situ hybridization, including peptide-nucleic-acid (“PNA”) FISH.

In certain embodiments, the above-described methods for detection via hybridization comprise the use of nucleic acids (e.g., FISH probes) comprising, or otherwise derived from, one or more viral genomes. In certain embodiments, the presently described methods are directed to the detection of viral genomes, and/or nucleic acids derived from viral genomes, of about 5,000 to 10,000 base pairs in length. For example, but not by way of limitation, the viral genome can be selected from any one of the genomes identified in Table 1.

TABLE 1
Representative GenBank Accession Numbers
for Complete Genomes of Viruses
(1) XMRV

| Strain | Accession # |
|---|---|
| VP35 | DQ241301 |
| VP42 | DQ241302 |
| VP52 | EF185282 |
| 22Rv1/CWR-R1 | FN692043 |
| RKO | JF274252 |
| xmlv15 | HQ154630 |
| S-162 | FR872816 |

(2) High-Risk HPV

| Type | Accession # |
|---|---|
| HPV-16 | FJ006723 |
| HPV-18 | AY262282 |
| HPV-26 | NC_001583 |
| HPV-31 | HQ537687 |
| HPV-33 | HQ537707 |
| HPV-35 | HQ537730 |
| HPV-39 | M62849 |
| HPV-45 | EF202167 |
| HPV-51 | M62877 |
| HPV-52 | HQ537751 |
| HPV-53 | GQ472849 |
| HPV-56 | EF177181 |
| HPV-58 | HQ537777 |
| HPV-59 | X77858 |
| HPV-66 | U31794 |
| HPV-68 | EU918769 |
| HPV-69 | AB027020 |
| HPV-73 | X94165 |
| HPV-82 | AF293961 |

(3) HIV-1

| | | Accession # |
|---|---|---|
| | Subtype | |
| Group M | A1 | AF004885 |
| | A2 | AF286240 |
| | B | K03455 |
| | C | U52953 |
| | D | K03454 |
| | F1 | AF077336 |
| | F2 | AJ249236 |
| | G | AF084936 |
| | H | AF190127 |
| | J | AF082384 |
| | K | AJ249235 |
| | L | AF286236 |
| | Strain | |
| Group O | ANT70 | L20587 |
| Group N | YBF106 | AJ271370 |
| Group P | RBF168 | GU111555 |

(4) HIV-2

| Subtype | Accession # |
|---|---|
| A | M30502 |
| B | X61240 |

(5) HTLV-1

| Strain | Accession # |
|---|---|
| 1067/05 | HQ808138 |

(6) HTLV-2

| Strain | Accession # |
|---|---|
| RP329 | AF326583 |

(7) HCV

| Genotype | Accession # |
|---|---|
| 1a | AB520610 |
| 1b | GU133617 |
| 2a | AF238485 |
| 2b | AF238488 |
| 3 | JN588558 |
| 4 | NC_009825 |
| 5 | NC_009826 |
| 6 | NC_009827 |

In certain embodiments, the above-described methods for detection via hybridization comprise the use of nucleic acids (e.g., FISH probes) comprising, or otherwise derived from, one or more viral genomes viruses or types of viruses: Parvoviruses (e.g., Parvovirus and Erythrovirus); Picornaviruses (e.g., Poliovirus, Rhinoviruses, Coxsackie viruses, and Hepatitis A); Flaviviruses (e.g., Dengue, Yellow Fever and West Nile); Papovaviruses (e.g., Polyomavirus); and Caliciviruses (e.g., Norovirus, Sapovirus, Lagovirus and Vesivirus).

The hybridization probes employed in the instantly described methods can be produced and labeled using techniques well known in the art. For example, but not by way of limitation, the probes can be produced via enzymatic or synthetic means and labeled by techniques such as, but not limited to, primer labeling and nick translation. A variety of production and labeling techniques, as well as hybridization and detection techniques that can be used in connection with the instant invention are detailed in Beatty et al. FISH: A Practical Approach, Oxford University Press, 2002.

In certain embodiments of the instant invention, the hybridization of the nucleic acid probe to the target viral nucleic acid occurs on a solid support. In the context of the present invention, a “solid support” refers to any such support that can be used for the methods of the invention. In certain embodiments of the present invention, the solid support is a transparent solid support. For example, but not by way of limitation, the solid support can be a microscope slide, a microfluidic chip, or a multi-well microplate. In certain embodiments, the solid support can be configured to receive one or more samples, e.g., one or more tissue samples. Such configurations can take any of a variety of forms (or combinations of forms), including, but not limited to, wells, grooves, pits, risers, or other indentations/protrusions.

In certain embodiments of the present invention, a nucleic acid probe will be contacted to a sample in order to assay whether that sample comprises a viral nucleic acid. In certain embodiments the sample will be a tissue specimen (e.g., formalin-fixed paraffin-embedded “FFPE” specimens), a bodily fluid (e.g., blood, plasma, serum, cerebrospinal fluid, saliva, tears, urine, or aqueous extracts of tissues and cells), or circulating tumor cells in blood. In certain, non-limiting, embodiments, the sample is contacted with a fixative, such as a buffered formalin solution or alcohol-based fixatives such as Carnoy's solution (methanol:acetic acid 3:1, v/v) either prior to, simultaneously with, or after placing the sample onto a solid support. For example, a sample, such as a blood sample, can be contacted with a buffered formalin solution prior to being placed onto a solid support, such that the nucleic acids present in the blood sample are fixed prior to the sample being placed on the solid support. While such fixation can be performed at any time, in certain embodiments of the present invention, the tissue sample is maintained at physiological salt (to prevent cell lysis) and pH conditions (to mimic normal body pH, and to maintain normal cell metabolic activity) until the sample is fixed. In alternative embodiments, the temperature of the sample is brought to about 4° C. to slow down metabolism. In additional alternative embodiments, the sample is frozen using methods that preserve cell viability, cell morphology, and other cell characteristics that are required for cell recognition, such as slow freezing in 10% DMSO.

In certain embodiments, the sample employed in the assay can be stored either in the fixative or on the solid support. In certain embodiments, the sample can be placed onto the solid support and then contacted with the buffered formalin solution, such that fixation of the nucleic acids present in the sample occurs after placement of the sample onto the solid support. In either case, the nucleic acid present in the sample is fixed and therefore stable until the hybridization assay is performed. In certain embodiments, the sample is de-proteinized, dehydrated, and/or rehydrated prior to hybridization using standard methods known to those in the art.

In certain embodiments, the hybridization assay of the present invention includes a nucleic acid denaturation step. For example, but not by way of limitation, a denaturation step can be included to reduce secondary structure of the hybridization probe and/or the nucleic acid target(s). In such embodiments, any method for denaturing nucleic acids can be used within the scope of the present invention. For example, in certain embodiments, the hybridization probe(s) and the target nucleic acid(s) are simultaneously denatured for between 30 seconds and one hour; between 30 seconds and 30 minutes; between 30 seconds and 10 minutes; between 30 seconds and 5 minutes; or between 1 and 2 minutes. In certain embodiments the denaturation is mediated by heating. In such embodiments, denaturation temperatures can be between 65°-100° C.; between 70° and 100° C.; or between 73° and 98° C. In alternative embodiments, denaturation is mediated by introduction of chemicals, such as detergents or other chaotropic compounds (e.g., urea, guanidinium), capable of interfering with normal hydrogen bonding of the hybridization probe or target nucleic acid. The concentrations and durations at which such compounds are employed depend on the size and composition of the sample(s) to be denatured and the determination of which is within the skill of one trained in the art.

In certain embodiments, the denaturation step is carried out in the solution that is used for the hybridization reaction. For example, in certain embodiments, a hybridization solution containing the hybridization probe(s) is applied to a tissue sample immobilized on a solid support and one or more coverslips, such as glass coverslips, are placed over the tissue sample-hybridization solution mixture to permit uniform spreading of the hybridization solution. In certain embodiments, such placement will occur with or without a sealant between the coverslip and the solid support. In certain embodiments, the nucleic acids in the sample (the hybridization probe(s) and those nucleic acids in the tissue sample) are initially denatured for about five minutes at an elevated temperature, such as 73° C., as described above. In certain embodiments hybridization between the probe(s) and the target nucleic acid occurs as the temperature decreases from the denaturation temperature to room temperature. In certain non-limiting embodiments, the decrease in temperature occurs by cycling through a series of 10 degree temperature increments, holding each temperature 10 seconds, through several cycles for each pair of temperatures. For example, but not by way of limitation, the sample can be cycled between about 80° C. and about 90° C. five times, maintaining each temperature for about 10 seconds, then between about 60° C. and about 70° C. for ten cycles (about 10 seconds each), then between about 50° C. and about 60° C. 10×, then about 30° C. and about 40° C. 10×, and finally about 25° C. and about 30° C. 10×. Alternatively, the samples can simply be brought to about 100° C. (+/− about 5° C.) for about 1 to about 2 minutes, then allowed to decrease steadily to about 55° C. over a period of about two to about five minutes, then kept at about 55° C. for about 30 minutes to overnight.

In certain embodiments, the assays of the present invention can be used in conjunction with any hybridization/wash buffers known in the art that are appropriate to carry out the relevant steps. The selection of particular buffers and conditions is well within the level of skill in the art. For example, in certain embodiments, the assays of the present invention utilize hybridization buffers as disclosed in U.S. Pat. Nos. 5,750,340 and 6,022,689. In certain of such embodiments, one of hybridization buffers F or G is used, as disclosed in U.S. Pat. No. 5,750,340: Buffer F: 10% (+/−2%) by weight dextran sulfate; 10%-30% by volume formamide; and 0.9% by weight of NaCl, KCl, or other appropriate salt. Buffer G: 10% (+/−2%) by weight dextran sulfate; 15-25% glycerol; 0.9% by weight of NaCl, KCl, or other appropriate salt.

Upon completion of the hybridization step, the sample is washed and screened for the presence of the hybridization probe(s). In certain embodiments, such post hybridization wash steps can incorporate a one or more wash solutions and one or more post-hybridization temperature conditions. For example, but not by way of limitation, the solid support carrying the sample can first be exposed to 2×SSC & 0.3% NP-40 for about 2 to about 5 minutes at room temperature in order to first float off the previously placed coverslip. After removal of the coverslip, the sample could be placed in the wash solution consisting of 0.3×-2×SSC & 0.3%-0.5% NP-40, and the temperature of the sample can be raised to about 73° C. for about 2-5 minutes. Then the support carrying the sample can be either counterstained with a nuclear DNA-binding stain, such as 4′,6-diamidino-2-phenylindole (DAPI) either in solution, or upon drying the sample in the dark. In the latter case, the sample is counterstained with about 10 μL DAPI, and a new coverslip is placed over the sample. The sample can then be viewed or stored, e.g., at about −20° C.

In certain embodiments, sample viewing involves detection of the hybridization probe(s). In certain embodiments, detection is facilitated by the presence of one or more labels operably linked to the hybridization probe(s). In certain embodiments the label is a direct label. Examples of direct labels include, but are no limited to, chromogens, radioisotopes (e.g., 125I, 131I, 32P, 3H, 35S, and 14C), fluorescent compounds (e.g., fluorescein and rhodamine), chemiluminescent compounds, and particles (visible or fluorescent). Additional fluorescent compounds include, but are not limited to: FCHA, ((Fluorescein-5-Carboxamido)Hexanoic Acid, Succinimidyl Ester (SpectrumGreen, SGn); DECCA, 7-diethylaminocoumarin-3-carboxylic acid, succinimidyl ester (SpectrumAqua, SA); TXRD, Texas Red (SpectrumRed, SR); CTMR, 5,6-carboxy-tetramethyl-rhodamine (SpectrumOrange, SO); and CR6G, 5-(and-6)-carboxyrhod-amine 6G (SpectrumGold, SGd).

In certain embodiments, such as, but not limited to, those embodiments where the hybridization probe is contacted to a population of cells, visualization of the probe can occur via fluorescent microscopy. In such embodiments, once the wash step is completed, the cells are stained in order to visualize individual fluorescent signals in individual cells. Such staining can include nuclear staining or other staining, such as with a fluorescent cell surface marker. In certain embodiments, the cells are counterstained with Hoechst 33342, 4,6-diamidino-2-phenylindole (DAPI) or propidium iodide (PI) solution. In certain embodiments, the fluorescent signal detection is further accompanied by identification of the cell in which the signal is detected. Such cell identification can be accomplished simultaneously with the nucleic acid fluorescent signal detection by, for example, dual hybridization with a fluorescent cell-specific surface antigen. Simultaneous cell identification can also be accomplished by co-hybridizing the hybridization probe with a second fluorescent probe for a cell-specific gene known to be expressed in the identified cell. In certain embodiments, the detection further comprises qualitative or quantitative analysis of the target nucleic present in individual cells. Fluorescence signals can be visualized with a fluorescence microscope equipped with a triple band-pass filter and a 20× or 40× dry objective lens. Signals can also be viewed at, e.g., 40×, 60×, or 100× under oil.

In certain embodiments, detection will involve interrogating a portion of the sample of interest. For example, but not by way of limitation, only a subset of the cells present in the sample prepared as outlined above will be analyzed for the presence of a signal indicative of the presence of the target nucleic acid(s). The identification of the portion of the sample that is to be analyzed can be the result of either intentional selection or random selection. In certain embodiments, portions of the sample will be intentionally selected based on a tissue-specific rationale, e.g., a particular virus can selectively infect certain tissues, thus the portion of the sample consisting of potentially infected tissue is interrogated. However, in certain embodiments, such as when samples comprise a single cell or tissue type, the selection of the portion of the sample to be interrogated does not need to be intentional selected.

In certain embodiments, the identification of a “positive” sample, that is a sample that is characterized as virally infected and/or indicative of a viral infection-associated disease state, does not require the identification of a signal in each and every cell, or even each and every virally-infected cell. For example, in certain embodiments, the identification of a single signal indicative of viral infection and/or indicative of a viral infection-associated disease state, is sufficient to identify a sample as testing “positive.” In certain embodiments, a sample will be designated “positive” if at least 0.1%-90% of the portion of the sample assayed displays a signal indicative of viral infection and/or indicative of a viral infection-associated disease state. In certain embodiments, a sample will be designated “positive” if at least 1%-50% of the portion of the sample assayed displays a signal indicative of viral infection and/or indicative of a viral infection-associated disease state. In certain embodiments, a sample will be designated “positive” if at least 5%-25% of the portion of the sample assayed displays a signal indicative of viral infection and/or indicative of a viral infection-associated disease state. In certain embodiments, a sample will be designated “positive” if at least 10%-20% of the portion of the sample assayed displays a signal indicative of viral infection and/or indicative of a viral infection-associated disease state.

In certain embodiments, the above-described sample preparation and analysis can be accomplished using commercially available hardware and software. For example, but not by way of limitation, the Vysis ThermoBrite® Denaturation/Hybridization unit and VP 2000 Processor (Abbott) HybRite, X-Matrix, Leica Bond, Ventana, or other FISH automation instruments and Imaging such as Ikonysis, BioView or MetaSystems or other imaging systems and associated software packages that can be used in the context of the instant invention. Such imaging systems have been designed to quantify individual signals, and they can accommodate the signal overlap that can occur in multi-FISH probe hybridization. These commercially available systems can also quantify the diffuse signals that occur in cytoplasmic nucleic acid hybridization as well as the discrete signals that occur in chromosomal hybridization.

### Combinations with Other FISH Markers & Personalized Medicine

In certain embodiments, one or more of the above-described molecular detection techniques can be combined with one or more additional chromosomal markers in the same assay. In certain embodiments, this is performed in order to simultaneously assess genomic abnormalities associated with a disease state, such as cancer. In certain embodiments, such combinations can be used to establish or confirm diagnosis, to assess prognosis (disease progression), or to stratify patients for therapy regimens. In certain embodiments, the combination of probes can be used for the diagnosis of cervical cancer. In certain of such embodiments, the combination contains a directly-labeled HPV probe combined with directly-labeled FISH probes for one, two, three, or more genomic loci including, but not limited to, TERC (3q26.2), TERT (5p15.33), and MYC (8q24.21).

A second non-limiting example of such a combined assay relates to the assessment of head and neck (H&N) cancers. For example, head and neck squamous cell carcinomas (HNSCCs) can arise in the oral cavity, oropharynx, larynx or hypopharynx. In a subgroup of HNSCCs, particularly those of the oropharynx, the disease is caused by infection with high-risk types of human papillomavirus (HPV). For example, HPV-16 in particular has been shown to be involved in HNSCC. Interestingly, tumors exhibiting HPV infection have been shown to be associated with a more favorable clinical outcome. (See, e.g., Gillison, M. L. et al. Evidence for a causal association between human papillomavirus and a subset of head and neck cancers. J Natl Cancer Inst. 92, 709-720 (2000)). In addition to HPV infection, H&N cancers are known to carry multiple genomic abnormalities in pathways responsible for cell proliferation. Many of these abnormalities were identified as genomic copy number changes that could be detected by FISH. (See, e.g., Leemans, C. R. et al. The molecular biology of head and neck cancer. Nature Reviews 11, 9-22 (2011)). Such copy number changes can provide additional diagnostic information (example—loss of p53), predict prognosis (example—loss of PTEN or p16), or provide assessment for molecular targets for targeted therapies (example—epithelial growth factor receptor (“EGFR”) inhibitors). Therefore, assays can be prepared using a multi-color probe set consisting of a combination of the HPV directly-labeled probe with one, two or three (or more depending on the circumstances) probes out of the directly-labeled FISH probes designed for the following genomic loci, given as a gene name and genomic location: EGFR (7p11.2), p53 (17p13.1), p16/CDKN2A (9p21), CCND1 (11q13.3), RB1 (13q14.2), TERT (5p15.33), MET (7q31.2), PIK3CA (3q26.32), TGFβ1 (19q13.2), MYC (8q24.21), PTEN (10q23.31) and DCC (18q21.2). In certain embodiments, each of these probes will represent a 100 kb to 800 kb genomic fragment centered on the gene of interest, with alternative embodiments directed to alternative size fragments, which are fragmented and directly labeled with fluorescent molecules of complimentary colors such that 1, 2, 3, 4, 5, or more probes are be detected in the same assay by visual evaluation and/or scanning/image capture.

For example, but not by way of limitation, EGFR is over-expressed in the majority of patients with head and neck squamous-cell carcinoma. Bonner et al., N Engl J Med. 2006; 354:567-578 and Bonner et al., Lancet Oncol. 2010; 11:21-28. In these studies it was shown that out of the four hundred twenty-four patients randomly assigned to receive RT alone or RT plus weekly cetuximab, a chimeric monoclonal antibody to the EGFR receptor, (where the majority of these patients had oropharynx primary tumors, and 75% of them received treatment with accelerated or hyperfractionated irradiation), the RT/cetuximab patients had significant improvements in median survival (49 months vs. 29 months; hazard ratio [HR]=0.73; p=0.02), and 5-year overall survival (46% vs. 36%). Subset analysis demonstrated the most pronounced therapeutic benefit in patients with oropharynx primaries, N-presentations, and treatment with accelerated fractionation. Thus, EGFR over-expression can be effectively used as a marker for HNSCC.

Similarly, it is appreciated that activation of the c-MET oncogene promotes tumor growth, invasion and metastasis in several tumor types. For example, c-MET pathways were examined in head and neck cancer and reported as a poster (#5520) from American Society of Clinical Oncology Annual meeting 2012. Casado, et al., J Clin Oncol 30, 2012 (suppl; abstr 5520). Casado et al. concluded that activation of the hepatocyte growth factor (“HGF”)/c-MET pathway correlated with less positive outcomes in cetuximab-based regimen treated HNSCC patients, where activation of the pathway acted as a resistance mechanism for EGFR inhibitors, supporting the concept of a dual block of HGF/c-MET and EGFR pathways in the treatment of these patients. Similar findings have been reported previously in lung and colorectal carcinomas in Appleman, J Clin Oncol. 2011; 29 (36):4837-4838. In addition, a paper by Catenacci et al. (Cancer Discovery 2011; 1:573-579) reported a biomarker of MET gene that could be used to predict therapeutic response to MET inhibition in a study of a 48-year-old woman. Thus, altered expression of c-Met can also be effectively used as a marker for HNSCC.

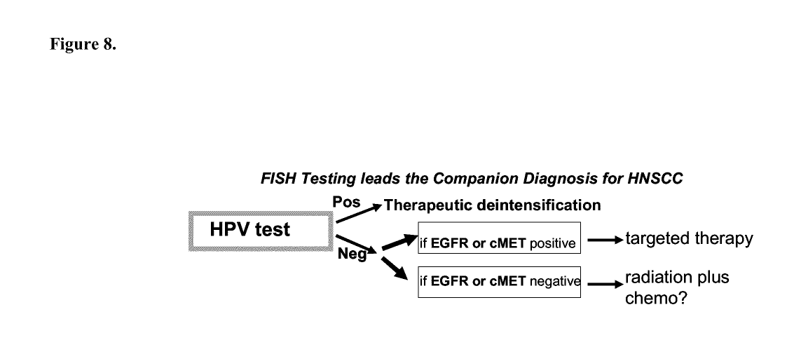

In light of the above-described studies, identification and validation of novel molecular markers as well as methods of assaying for those molecular markers, e.g., the methods outlined in the present application, can assist in determining patient prognosis, drug therapy selection, as well as diagnostic testing for head and neck squamous cell carcinoma. In certain non-limiting embodiments, such methods include hybridizing one or more chromosomal probes to a biological sample obtained from a subject (HNSCC) and recording the hybridization pattern of the chromosomal probes and status of HPV infection to determine differences in the hybridization profile. These methods also have utilities in patient surveillance, as an aid in cancer risk assessment, as well as in drug therapy selection as “personalized medicine” for HNSCC patients. For example, but not by way of limitation, the use of 1, 2, 3, 4, or more FISH probes for genetic analysis of HNSCC specimens can permit the stratification of patients with regard to prognosis, as well as assisting in drug therapy selection (i.e., use as a companion diagnostic). One exemplary algorithm for HNSCC patient stratification is outlined in FIG. 8.

Combinations With Alternative Detection Techniques

In certain embodiments, one or more of the above-described molecular detection techniques can be combined with one or more alternative detection techniques. For example, but not by way of limitation, one or more of the above-described molecular detection techniques can be performed in concert with, e.g., prior to, in conjunction with, or after, the performance of an alternative detection technique. In certain embodiments, the alternative detection technique is an immunoassay.

There are two basic types of immunoassays, competitive and non-competitive (e.g., immunometric and sandwich, respectively). In both assays, antibody or antigen reagents are covalently or non-covalently attached to the solid phase. (See The Immunoassay Handbook, 2nd Edition, edited by David Wild, Nature Publishing Group, London 2001.) Linking agents for covalent attachment are known and can be part of the solid phase or derivatized to it prior to coating. Examples of solid phases used in immunoassays are porous and non-porous materials, latex particles, magnetic particles, microparticles, strips, beads, membranes, microtiter wells and plastic tubes. The choice of solid phase material and method of labeling the antigen or antibody reagent are determined based upon desired assay format performance characteristics. For some immunoassays, no label is required. For example, if the antigen is on a detectable particle such as a red blood cell, reactivity can be established based upon agglutination. Alternatively, an antigen-antibody reaction can result in a visible change (e.g., radial immunodiffusion). In most cases, one of the antibody or antigen reagents used in an immunoassay is attached to a signal-generating compound or “label”. As described above, the signal-generating compound or “label” is in itself detectable (“direct labeling”) or can be reacted with one or more additional compounds to generate a detectable product (“indirect labeling”; see also U.S. Pat. No. 6,395,472 B1).

There are three general formats commonly used to monitor specific antibody titer and type in humans: (1) the indirect anti-human assay format, where antigen is presented on a solid phase, as described above, the human biological fluid containing the specific antibodies is allowed to react with the antigen forming an antigen/antibody complex, and then antibody bound to antigen is detected with an anti-human antibody coupled to a signal-generating compound; (2) the semi-direct anti-human assay format, where an anti-human antibody is bound to the solid phase, the human biological fluid containing specific antibodies is allowed to react with the bound anti-human antibody forming an anti-human antibody/antibody complex, and then antigen attached to a signal-generating compound is added to detect specific antibody present in the fluid sample, and (3) the direct double antigen sandwich assay format, where antigen is presented both as capture antigen and as detection conjugate, as described in format (1), antigen is presented on a solid phase, the human biological fluid containing the specific antibodies is allowed to react with the antigen bound on solid phase forming an antigen/antibody complex, and then antibody bound to antigen is detected with the antigen coupled to a signal-generating compound. In formats (1) and (2), the anti-human antibody reagent can recognize all antibody classes, or alternatively, be specific for a particular class or subclass of antibody, depending upon the intended purpose of the assay.

Format (3) has advantages over formats (1) and (2) in that it detects all antibody classes and antibodies derived from all mammalian species. These assay formats as well as other known formats are intended to be within the scope of the present invention and are well-known to those of ordinary skill in the art

### Diagnostic Methods And Kits

In certain embodiments, the present invention provides methods for detecting viral nucleic acids that are indicative of viral infection, and/or prognosis of viral infection and associated disease states. In certain embodiments, the methods of the present invention are directed to detecting viral infections such as, but not limited to, XMRV infection, HIV-1 infection, HIV-2 infection, HCV infection, HTLV-1 infection, HTLV-2 infection, and HPV infections.

In certain embodiments, the present invention provides methods for detecting viral-associated disease states such as Acquired Immunodeficiency Syndrome (AIDS) and AIDS-related diseases, hepatocellular carcinoma, adult T-cell leukemia, hairy cell leukemia as well as cancers of the prostate, cervix, uterus, anus, oropharynx, penis, vagina, and vulva. In certain embodiments the present invention provides methods for detecting viral nucleic acids that are indicative of a propensity to develop Acquired Immunodeficiency Syndrome (AIDS) and AIDS-related diseases, hepatocellular carcinoma, adult T-cell leukemia, hairy cell leukemia as well as cancers of the prostate, cervix, uterus, anus, oropharynx, penis, vagina, and vulva.

In certain embodiments, the present invention provides methods for the detection of viral nucleic acid molecules within cells, including DNA and/or RNA molecules. For example, but not by way of limitation, viral RNA can be detected by FISH DNA probes. In certain of such embodiments, a DNA probe specific to the full-length viral genome is directly labeled with SpectrumOrange (carboxytetramethylrhodamine, CTMR) fluorophore molecule, where this molecule is chemically attached to cytosine bases via an aminoethyl linker.

In certain embodiments, the present invention also provides for diagnostic tests capable of detecting viral nucleic acids in a variety of sample types. For example, but not by way of limitation, an assay of the present invention can interrogate white blood cells, cytology specimens (e.g., urine, cervical brushing and swab specimens, esophageal brushing specimens, fine-needle aspirates, saliva, among others), and tissue specimens (such as archived FFPE tissue specimens).

In certain embodiments, the techniques described herein provide for the identification of viral DNA via treatment with the RNase enzyme, and the identification of different functional states of the virus will be employed in the context of oncology. For example, but not by way of limitation, the detection of total viral nucleic acids (DNA and RNA) can be used as a diagnostic test for cancers known for association with a specific viral species. The detection of total viral nucleic acids (DNA and RNA) can also be used as a prognostic test for cancer.

In certain embodiments, the present invention provides methods for detecting viral infection that additionally incorporate the use of one or more alternative molecular detection technique, e.g., LCR, SDA, RT-PCR, or NASBA. Such embodiments can additionally incorporate one or more immunodetection technique, including, but not limited to, direct or indirect immunoassays, such as direct or indirect ELISA assays. In certain embodiments, the present invention provides methods for detecting viral infection and/or associated disease states that involve the use of one or more molecular detection technique, e.g., FISH LCR, SDA, RT-PCR, or NASBA, in the context of assaying a panel of viral infection and/or associated disease state markers. Such panels can include one or more markers of XMRV infection and/or associated disease state.

In certain embodiments, markers employed in the context of the methods of the present application include, but are not limited to, markers of chromosomal instability at a numerical or structural level, such as aneuploidy of chromosome regions 3q26 (TERC)5-7 and 8q24 (MYC)8-10 in cervical malignant and pre-malignant lesions and cervical cancer, elevated PSA levels, prostate cancer-specific gene expression (See, e.g., Bradford et al., Molecular markers of prostate cancer, Urol. Oncol. 24(6), 538-551 (2006)), cervical cancer-specific gene expression (See. e.g., Bachtiary et al., Gene expression profiling in cervical cancer: an exploration of intratumor heterogeneity, Clin Cancer Res 12:5632-5640 (2006)), uterine cancer-specific gene expression (See, e.g., Smid-Koopman et al., Gene expression profiling in human endometrial cancer tissue samples: utility and diagnostic value, Gynecologic Oncology 93(2): 292-300 (2004)), and chronic fatigue syndrome-specific gene expression (See, e.g., Fletcher et al., Biomarkers in Chronic Fatigue Syndrome: Evaluation of Natural Killer Cell Function and Dipeptidyl Peptidase IV/CD26. PLoS ONE 5(5): e10817 (2010)). In certain embodiments, the present invention provides methods for detecting a propensity to develop prostate cancer, cervical cancer, uterine cancer, or chronic fatigue syndrome that involve the use of one or more viral infection molecular detection technique in the context of assaying a panel of prostate cancer, cervical cancer, uterine cancer, or chronic fatigue syndrome markers.

A positive result using any of the above-described methods, indicative of the presence of viral infection and/or an associated disease state, can optionally be followed by a corroborative or confirmative diagnostic procedure, such as but not limited to, an immunoassay, a tissue biopsy, a histologic evaluation, a radiographic study, a MRI study, an ultrasound study, a PET scan, etc.

Any of the exemplary amplification-based assay formats described herein and any assay or kit according to the invention can be adapted or optimized for use in automated and semi-automated systems (including those in which there is a solid phase comprising a microparticle), as described, e.g., in U.S. Pat. Nos. 5,089,424 and 5,006,309, and as, e.g., commercially marketed by Abbott Laboratories (Abbott Park, Ill.) including but not limited to Abbott's ARCHITECT®, AxSYM, IMX, PRISM, m2000, and Quantum II platforms, as well as other platforms.

The assays and kits of the present invention optionally can be adapted or optimized for point of care assay systems. For example, but not by way of limitation, the in situ hybridization assays can be performed according to the methods described in WO 2008031228, entitled “Automated Fish Analysis, Circulating Microfluidic Chip, And Method For Immobilizing Cells To A Microfluidic Chip.” In addition, for those embodiments comprising an immunoassay component, such assays can also be adapted for point of care assay systems, such as Abbott's Point of Care (i-STAT™) electrochemical immunoassay system. Immunosensors and methods of manufacturing and operating them in single-use test devices are described, for example in U.S. Pat. No. 5,063,081 and published U.S. Patent Application Publication Nos. 20030170881, 20040018577, 20050054078, and 20060160164.

Diagnostic kits are also included within the scope of the present invention. More specifically, the present invention includes kits for determining the presence of viral nucleic acids in a test sample. In certain embodiments of the present invention, a kit for detection of viral nucleic acids comprises: (1) a nucleic acid sequence comprising a target-specific sequence that hybridizes specifically to an viral nucleic acid target, and (2) a detectable label. In certain embodiments, the target-specific sequence (probe) is directly-labeled with the detectable label.

In certain embodiment, the kits of the invention are useful for detecting multiple viral nucleic acid targets. In such situations, the kits can comprise, for each different nucleic acid target, a distinct probe nucleic acid and one or more distinct labels. In certain embodiments, the kit will comprise a directly-labeled probe capable of hybridizing to a viral sequence from a virus having a genome of less than 10,000 base pairs and one or more additional probes capable of specifically binding to an alternative target sequence, and the additional probe or probes can be directly-labeled or can make use of indirect or amplification-based labels.

In certain embodiments the kit comprises nucleic acids (e.g., hybridization probes) comprising or otherwise derived from an XMRV viral genome, including, but not limited to, one or more of the sequences identified in Table 1.

In certain embodiments the kit comprises nucleic acids (e.g., hybridization probes) comprising or otherwise derived from an HPV viral genome, including, but not limited to the viral genomic sequences of one or more of the following HPV types: HPV-16; HPV-18; HPV-26; HPV-31; HPV-33; HPV-35; HPV-39; HPV-45; HPV-51; HPV-52; HPV-53; HPV-56; HPV-58; HPV-59; HPV-66; HPV-68; HPV-69; HPV-73; and HPV-82 as identified in Table 1.

In certain embodiments the kit comprises nucleic acids (e.g., hybridization probes) comprising or otherwise derived from an HIV viral genome, including, but not limited to the viral genomic sequences of one or more of the following HIV variants: HIV-1 and HIV-2 as identified in Table 1.

In certain embodiments the kit comprises nucleic acids (e.g., hybridization probes) comprising or otherwise derived from an HTLV viral genome, including, but not limited to the viral genomic sequences of one or more of the following HTLV variants: HTLV-1 and HTLV-2 as identified in Table 1.

In certain embodiments the kit comprises nucleic acids (e.g., hybridization probes) comprising or otherwise derived from an HCV viral genome, including, but not limited to the viral genomic sequences of one or more of the HCV genotypes identified in Table 1.

In certain embodiments the kit comprises nucleic acids (e.g., hybridization probes) comprising or otherwise derived from a genome of one or more of the following viruses or types of viruses: Parvoviruses (e.g., Parvovirus and Erythrovirus); Picornaviruses (e.g., Poliovirus, Rhinoviruses, Coxsackie viruses, and Hepatitis A); Flaviviruses (e.g., Dengue, Yellow Fever and West Nile); Papovaviruses (e.g., Polyomavirus); and Caliciviruses (e.g., Norovirus, Sapovirus, Lagovirus and Vesivirus).

The present invention can be illustrated by the use of the following non-limiting examples.

### EXAMPLES

### Example 1

### Direct Labeling Procedure for SpectrumOrange Labeled XMRV FISH Probe (XMRV-SO)

DNA of a plasmid clone VP62/pcDNA3.1 having a full-length (˜8.2 kb) XMRV VP62 genome (Dong et al., Proc Natl Acad Sci USA 104:1655-1660 (2007)) was extracted using PureLink MaxiPrep DNA kit (Invitrogen, Carlsbad, Calif.) according to the manufacturer's instructions.

To 200 μg of the entire plasmid DNA (˜13.6 kb), water was added to 360 μl which was then mixed with 40 μl of 3 M sodium acetate. DNA solution was placed on ice and sonicated for 2×6 min on setting 2 (20% duty cycle) with vortexing briefly between sonications. Sonicated DNA was extracted first with 300 μl of phenol and then with another 300 μl of chloroform:isobutanol (24:1). The mixture was partitioned by centrifuging for 5 min at room temperature. The upper aqueous layer containing DNA was transferred to a new tube. The lower organic layer was extracted with 400 μl of 0.3 M sodium acetate, and then centrifuged for 15 min at room temperature to obtain another upper aqueous layer that was transferred and pooled with the first upper layer. Pooled DNA solution was mixed with 2.5 volumes of 100% ethanol and precipitated at −20° C. overnight. Precipitated DNA was collected by centrifuging at 13,000 rpm for 15 min at 4° C. DNA pellets were washed with 400 μl of 70% ethanol, dried in a speedvac and resuspended in a combined volume of 80 μl water.

An amination mix of 1 ml water, 600 μl TFA (trifluoroacetic acid), 348 μl ED (ethylenediamine), and 190 mg sodium metabisulfite, pH 6.83, was freshly prepared. 20 μl of sonicated plasmid DNA as prepared above was heated in a boiling water bath for 5 min and then mixed with 180 μl of amination mix. The DNA mixture was incubated at 65° C. for 35 min and placed on ice. Aminated DNA was desalted using a Sephadex G25 column and collected in 400 μl water. 40 μl of 3 M sodium acetate and 1 ml of 100% ethanol were added to precipitate DNA at −20° C. overnight. Precipitated DNA was collected by centrifuging at 13,000 rpm for 25 min at 4° C. DNA pellets were washed with 400 μl of 70% ethanol, dried in a speedvac and resuspended in a combined volume of 40 μl water.

To 40 μl of aminated DNA in water, 8 μl of 50 mM SpectrumOrange fluorophore TAMARA-SE (5-(and-6)-carboxytetramethylrhodamine, succinimidyl ester 5(6)-TAMRA, SE) in DMSO was added. The mixture was placed in a 60° C. oven for 30 min to covalently attach the fluorophore to the aminated DNA. Then, another 8 μl of the 50 mM fluorophore solution was added into the DNA mixture, and the mixture was returned to the 60° C. oven for additional one hour. The labeled DNA was separated from the unincorporated fluorophores by undergoing an ethanol precipitation with the addition of 200 μl water, 30 μl 3M sodium acetate and 900 μl 100% ethanol at −20° C. for one hour. Precipitated DNA was collected by centrifuging at 13,000 rpm for 10 min at 4° C. DNA pellets were washed with 400 μl of 70% ethanol, dried in a speedvac and resuspended in a combined volume of 50 μl water containing 5 μl of 1 M NaOH. The labeled DNA probe was further desalted through a Sephadex G25 column and collected in 400 μl water. The concentration of purified SpectrumOrange labeled XMRV DNA probe (XMRV-SO) was determined by measuring OD₂₆₀ using a NanoDrop spectrophotometer. The percentage of SpectrumOrange incorporation in the XMRV-SO probe was determined to be approximately 8.0%.

### Example 2

### Growth and Harvest of Human Prostate Cancer Cell Lines (DU145 and 22Rv1) for XMRV FISH Assays

DU145 cells (ATCC #: HTB-81) uninfected and producing no XMRV (Dong et al., Proc Natl Acad Sci USA 104:1655-1660 (2007)), and 22Rv1 cells (ATCC #: CRL-2505) carrying at least 10 integrated copies of XMRV and generating high-titer XMRV virus (Knouf et al., J Virol 83:7353-7356 (2009)), were used as a negative and positive control, respectively, in XMRV FISH assays. These cells were propagated in DMEM-F12 complete medium (Invitrogen) at 37° C. in an atmosphere of 5% carbon dioxide. After growing to a 60% to 70% confluence level, cells were arrested with 1 ml of Colcemid solution (10 μg/ml) (Invitrogen) for every 50 ml cell culture and incubated at 37° C. for 2 hours. Cells were then harvested using a standard trypsinization procedure. After washing collected cells once with 40 ml of 1×DPBS (Invitrogen), cells were resuspended in 40 ml of 0.075 M potassium chloride solution (Invitrogen) and incubated at 37° C. for 30 min. Cells were subsequently washed 4 times each with 40 ml of Carnoy's fixative (methanol:glacial acetic acid=3:1 (v/v)) (Fisher, Pittsburgh, Pa.), and were finally resuspended in 5 ml of Carnoy's fixative and stored at −20° C. Slides having a mixture of DU145 and 22Rv1 were prepared by depositing cells (10 μl per slide for each of the two cell suspensions) on SuperFrost Plus positively charged slides (ThermoShandon, Pittsburgh, Pa.). Cell-coated slides were then air-dried overnight at room temperature before subjected to pretreatment and hybridization.

### Example 3

### Specimen Pretreatment and In Situ Hybridization Using XMRV-SO Probe

Slides containing a mixture of DU145 and 22Rv1 cells were pretreated in 2×SSC (0.3 M NaCl, 0.03 M sodium citrate, pH 7.0 (Invitrogen)) at 73° C. for 2 min followed by a 10-min incubation in pepsin solution (USB, Cleveland, Ohio) (0.5 mg/ml in 10 mM HCl) at 37° C. Slides were then at room temperature rinsed in 1×DPBS (Invitrogen) for 5 min, fixed in a 1% neutral-buffered formalin solution (Fisher) for 5 min and rinsed again in 1×DPBS for another 5 min. Dehydration of slides was performed through a series of ethanol: 1 min each in 70%, 85%, and 100%, followed by air-drying. Slides were ready for hybridization with the XMRV-SO probe.

If digestion of RNA was required, slides containing a mixture of DU145 and 22Rv1 cells were pretreated in 2×SSC (pH 7.0) at 73° C. for 2 min, and incubated for 1 hour at 37° C. in 2×SSC (pH 7.0) containing 100 μg/ml RNase A (Sigma, St. Louis, Mo.). After RNase A pretreatment, slides at room temperature were washed four times in 2×SSC (pH 7.0) for 5 min each, and once in water for 2 min. Slides were incubated in pepsin solution (0.5 mg/ml in 10 mM HCl) at 37° C. for 10 min. Slides were then at room temperature rinsed in 1×DPBS (Invitrogen) for 5 min, fixed in a 1% neutral-buffered formalin solution (Fisher) for 5 min and rinsed again in 1×DPBS for another 5 min. Dehydration of slides was performed through a series of ethanol: 1 min each in 70%, 85%, and 100%, followed by air-drying. Slides were ready for hybridization with the XMRV-SO probe.

A 10 μl of probe hybridization mix was prepared by mixing 100 ng XMRV-SO, 100 ng CEP8-SA, 1000 ng sonicated human placental DNA, 250 ng human Cot-1 DNA, and 7 μl LSI/WCP hybridization buffer (Abbott Molecular, Inc., Des Plaines, Ill.). Ten microliters of the probe mix were added to the cell line specimens, slides were coverslipped (22×22 mm) (VWR, Radnor, Pa.), and sealed with rubber cement (Staples, Framingham, Mass.). Probes and specimen nucleic acids on each slide were co-denatured for 3 min at 73° C. and immediately hybridized for 16-24 hours at 37° C. on a ThermoBrite (Abbott Molecular, Inc.). Following hybridization, coverslips were removed, and slides were washed in 0.4×SSC/0.3% NP-40 (Abbott Molecular, Inc.) at 73° C. for 2 min and then in 2×SSC/0.1% NP-40 (Abbott Molecular, Inc.) for 1 min at room temperature. 10 μl of DAPI II (125 ng/ml) (Abbott Molecular, Inc.) counterstain was placed on the slide and a coverslip was applied.

All slides mounted with FFPE human prostate cancer tissue sections were baked at 56° C. for 4 hours to fix the tissue onto the slides and were then stored at room temperature. In preparation for in situ hybridization, tissue specimen slides at room temperature were deparaffinized by soaking in three changes of Hemo-De solvent (Scientific Safety Solvents, Keller, Tex.) for 5 min each, followed by two 1-minute rinses in 100% ethanol, an incubation in a solution of 45% formic acid (Fisher)/0.3% hydrogen peroxide (Calbiochem, San Diego, Calif.) for 15 min, and a rinse in deionized water for 3 min. Slides were then immersed in pretreatment solution (Abbott Molecular, Inc.) at 80° C. for 35 min, rinsed for 3 min in deionized water at room temperature, incubated for 22 min in pepsin solution (1.5 mg/ml in 0.1 N HCl) at 37° C., and rinsed again for 3 min in deionized water at room temperature. Slides were subjected to dehydration at room temperature for 1 min each in 70%, 85%, and 100% ethanol, and were then air-dried. A 10 μl of probe hybridization mix was prepared by mixing 100 ng XMRV-SO, 100 ng CEP8-SA, 1000 ng sonicated human placental DNA, 250 ng human Cot-1 DNA, and 7 μl LSI/WCP hybridization buffer. Ten microliters of the probe mix were added to the tissue specimens, slides were coverslipped, and sealed with rubber cement. Probes and tissue specimen nucleic acids on each slide were co-denatured for 5 min at 73° C. and immediately hybridized for 16-24 hours at 37° C. on a ThermoBrite. Following hybridization, slides were soaked in 2×SSC/0.1% NP-40 at room temperature until coverslips came off, then washed in 0.4×SSC/0.3% NP-40 at 73° C. for 2 min, and subsequently in 2×SSC/0.1% NP-40 for 1 min at room temperature. 10 μl of DAPI I (1,000 ng/ml) (Abbott Molecular, Inc.) counterstain was added on the slide and a coverslip was applied.

After adding nuclear counterstain DAPI, slides were evaluated using fluorescence microscopy with DAPI, SpectrumOrange, and SpectrumAqua fluorescence filters (Abbott Molecular, Inc.).

### Example 4

### Results of XMRV FISH Assays Using XMRV-SO Probe

XMRV probes specifically hybridize to XMRV viral sequence (DNA or RNA) to detect virus-infected cells and to provide information about the location (nucleus or cytoplasm) of viral sequence in an infected cell. CEP8-SA probe hybridizes to the centromere region of human chromosome 8 to serve as an internal control to monitor the FISH hybridization step and to provide a tool for identification of the copy number of chromosome 8 by enumerating the number of aqua colored probe signals.

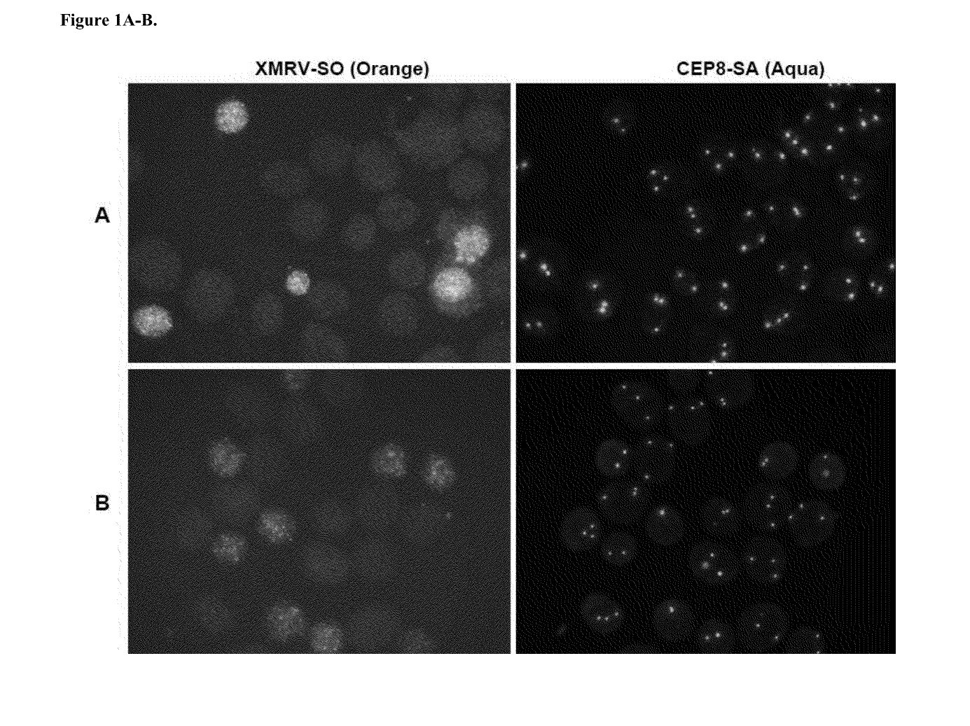

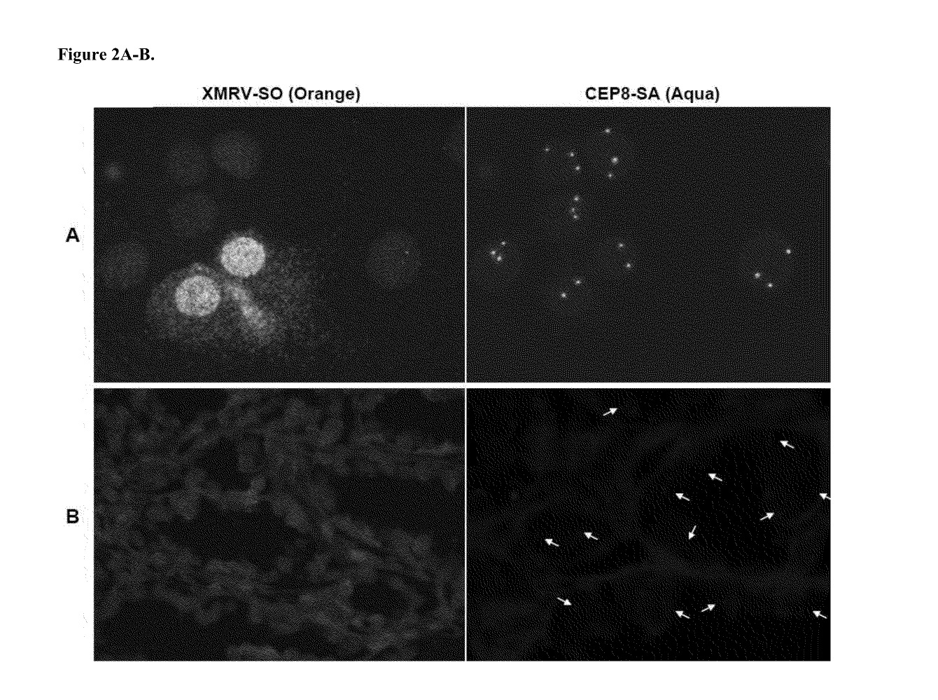

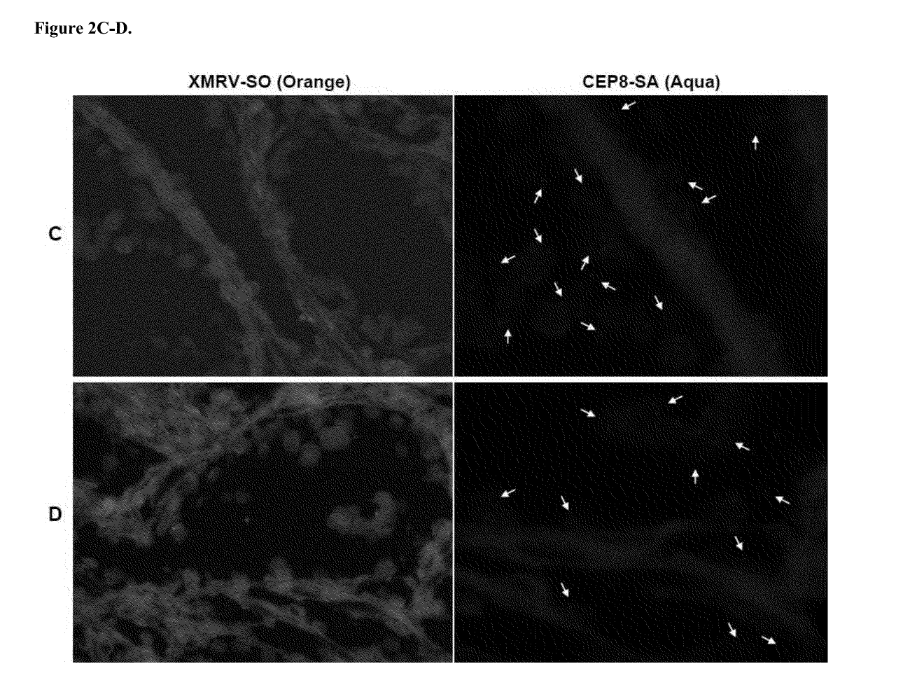

Results of XMRV FISH assays using XMRV-SO probe in human prostate cancer cell lines DU145 (negative control) and 22Rv1 (positive control) as described in Examples 2 amd 3 are shown in FIG. 1 (A-B) and FIG. 2 (A). As presented in FIG. 1 (A) and FIG. 2 (A), hybridization to the directly-labeled XMRV-SO probe was only observed for the 22Rv1 cells. The XMRV orange staining in 22Rv1 was intensely localized to the nuclei, while some staining was found in the cytoplasm. The chromosomal control probe (CEP8-SA) stained both cell types and readily distinguished DU145 (3 aqua signals) from 22Rv1 (2 aqua signals). Pretreatment of DU145 and 22Rv1 cells with RNase A to digest both cellular and viral RNA prior to hybridization with XMRV-SO generated a punctate staining pattern with multiple bright orange spots in every single 22Rv1 cell nucleus but not in DU145 as shown in FIG. 1 (B). A punctate staining pattern is indicative of the presence of integrated XMRV proviral DNA. These results demonstrate that the XMRV-SO probe is capable of directly and specifically detecting integrated XMRV DNA in the nucleus, and XMRV RNA in both nucleus and cytoplasm.

Examination of the FFPE prostate cancer tissue sections of 28 patients (Cleveland Clinic, Cleveland, Ohio; Rush University Medical Center, Chicago, Ill.) by FISH revealed no evidence of XMRV infection. All specimens were uniformly negative (representative fields shown in FIG. 2, B-D) with the XMRV-SO probe although the internal control probe CEP8-SA signal was observed, indicating fidelity of the hybridization process. These negative results are in good agreement with a growing number of reports showing that there is no evidence of XMRV in prostate cancer (Hohn et al., Retrovirology 6:92 (2009); Sakuma et al., Retrovirology 8:23 (2011); Switzer et al., PLoS One 6(5):e19065 (2011); Akgül et al., Med Microbiol Immunol Sep. 6 (2011); Stieler et al., PLoS One 6(10):e25592 (2011)).

### Example 5

### Direct Labeling Procedure for SpectrumOrange Labeled HPV-16 FISH Probe (HPV16-SO)

A plasmid DNA containing a full-length genome of HPV-16 (˜8.0 kb) (Sokolova et al., J Mol Diagn 9:604-611 (2007)) was isolated and purified as described in Example 1. 200 μg of purified plasmid was used to undergo amination and then labeling with SpectrumOrange fluorophore TAMRA as described in Example 1. The HPV16-SO FISH probe was 8.5% labeled with SpectrumOrange fluorophore.

### Example 6

### FISH Assays Using the HPV16-SO Probe for Detection of HPV in a Human Cervical Cancer Cell Line (CaSki)

The CaSki cell line (ATCC #: CRL-1550) was derived from a patient with an epidermoid carcinoma of the cervix (Pattillo et al., Science 196:1456 (1977)). The cell line is reported to contain ˜600 copies of integrated human papillomavirus type 16 genome (HPV-16) per cell (Yee et al., Am J Pathol 119:361-366 (1985); Pater and Pater, Virology 145:313-318 (1985); Lizard et al., Diagn Cytopathol 24:112-116 (2001)). CaSki positive control slides from the Vysis Cervical FISH Probe Kit (Abbott Molecular Inc., Des Plaines, Ill.) were used for the analysis. As negative controls, 10 μl of each of the two prostate cancer cell suspensions (DU145 and 22Rv1 in Example 2) were dropped onto a microscope slide and air-dried overnight at room temperature. Prior to hybridization, the two cell-laden slides (CaSki and DU145/22Rv1) were incubated at 73° C. for 2 min in 2×SSC (pH 7.0), digested at 37° C. for 10 min with 0.5 mg/ml pepsin in 0.01 N HCl, and washed with 1×DPBS for 5 min at room temperature. The protease-digested cells were then fixed onto slides for 5 min at room temperature in 1% formaldehyde (12.5 ml of 10% neutral buffered formalin, 36.5 ml of 1×DPBS and 1 ml of 1 M MgCl₂), and excess formaldehyde was washed off the slides with 1×DPBS at room temperature for 5 min. The washed slides were then passed through a series of three ethanol steps (70%, 85% and 100%) at room temperature for 1 min each. After dehydration, the slides were air-dried at room temperature and ready for hybridization with the HPV16-SO probe.

Ten μl of HPV16-SO probe mix was prepared by mixing 100 ng HPV16-SO probe, 100 ng CEP8-SA probe (an internal control probe for human chromosome 8), 1000 ng sonicated human placental DNA, 250 ng human Cot-1 DNA, and 7 μl LSI/WCP hybridization buffer. Ten μl of the probe mixture was then applied on the target area of the slide. A 22×22 mm coverslip was placed to cover the hybridization mixture and sealed with rubber cement. The probes and sample nucleic acids on the slide were co-denatured at 73° C. for 3 min and then hybridized at 37° C. for 16-24 hours on a ThermoBrite. After hybridization, the coverslip was removed, and the slide was washed at 73° C. for 2 min in 0.4×SSC/0.3% NP-40, followed by 2×SSC/0.1% NP-40 for 1 min at room temperature. After washing, the slide was air-dried at room temperature, applied with 10 μl of nuclear counterstain DAPI II (125 ng/ml) and covered with a new 22×22 mm coverslip. The stained slide was ready for an evaluation under a fluorescent microscope or stored at −20° C. for future evaluation

### Example 7

### Using a Human Cervical Cancer Cell Line (SiHa) to Examine the Sensitivity of the HPV16-SO Probe for Detecting HPV DNA in FISH

The SiHa cell line (ATCC #: HTB-35) was derived from a patient with a grade II squamous cell carcinoma of the cervix (Friedl et al., Proc Soc Exp Biol Med 135:543-545 (1970)). The cell line is reported to contain 1 to 2 copies of integrated human papillomavirus type 16 genome (HPV-16) per cell (Yee et al., Am J Pathol 119:361-366 (1985); Pater and Pater, Virology 145:313-318 (1985); Lizard et al., Diagn Cytopathol 24:112-116 (2001)). The SureDetect SiHa Cell Control supplied in a buffered ethanol-based preservative fluid was purchased from TriPath Imaging, Inc. (Burlington, N.C.) and used to test the sensitivity of the HPV16-SO FISH probe. The SiHa Cell Control was packaged in a dropper-tip bottle, and three drops of the cell suspension according to the package insert were applied onto a microscope slide and air-dried overnight at room temperature. Prior to hybridization, the cell-laden slide (SiHa) was incubated at 73° C. for 2 min in 2×SSC (pH 7.0), digested at 37° C. for 10 min with 0.5 mg/ml pepsin in 0.01 N HCl, and washed with 1×DPBS for 5 min at room temperature. The protease-digested cells were then fixed onto the slide for 5 min at room temperature in 1% formaldehyde (12.5 ml of 10% neutral buffered formalin, 36.5 ml of 1×DPBS and 1 ml of 1 M MgCl₂), and excess formaldehyde was washed off the slide with 1×DPBS at room temperature for 5 min. The washed slide was then passed through a series of three ethanol steps (70%, 85% and 100%) at room temperature for 1 min each. After dehydration, the slide was air-dried at room temperature and ready for hybridization with HPV16-SO probe. The hybridization was performed as described in Example 6.

### Example 8

### FISH Assays Using HPV16-SO Probe for Detection of HPV in a FFPE Human Cervical Cancer Tissue Specimen

HPVs are small, non-enveloped viruses that contain circular double-stranded DNA genomes of approximately 8.0 kb (Yugawa and Kiyono, Rev Med Virol 19:97-113 (2009)). Over 100 different genotypes of HPVs have been identified, of which about 40 infect the genital mucosa. HPV-16, 18 and 31 are reported to be associated with more than 90% of cervical cancers. HPV genomes replicate episomally in host cells, but HPV DNA is frequently found to be integrated into chromosomes in cervical cancer cells. To test the capability of HPV16-SO in detecting the HPV-infected cells in human tissue, a slide having an FFPE human cervical cancer tissue section (V02-90) (University of Texas Southwestern Medical Center, Dallas, Tex.) was used. This specimen V02-90 was identified as HPV-positive using a biotin-labeled HPV probe set in combination of tyramide signal amplification (Vysis Cervical FISH Probe Kit, Abbott Molecular, Inc.). As a negative control, an FFPE human prostate cancer tissue section (26604) (Rush University Medical Center, Chicago, Ill.) was used to undergo the same testing with HPV16-SO.

The two FFPE tissue section slides were baked at 56° C. for 4 hours and then stored at room temperature. Prior to hybridization, the slides were deparaffinated three times in Hemo-De solvent at room temperature for 5 min each, after which they were washed twice with 100% ethanol at room temperature for 1 min each. After washing, the slides were incubated at 80° C. for 35 min in 1×SSC (pH 6.3), washed with water for 3 min at room temperature, digested with 1.5 mg/ml of pepsin in 0.1 N HCl at 37° C. for 22 min, and washed again with water for 3 min at room temperature. The slides then went through a series of three ethanol steps (70%, 85% and 100%) at room temperature for 1 min each. After dehydration, the slides were air-dried at room temperature and ready for hybridization with the HPV 16-SO probe.

Ten μl of the HPV16-SO probe mix was prepared by mixing 100 ng HPV16-SO probe, 100 ng CEP8-SA probe, 1000 ng sonicated human placental DNA, 250 ng human Cot-1 DNA, and 7 μl LSI/WCP hybridization buffer. Ten μl of the probe mixture was then applied on the target area of the slide. A 22×22 mm coverslip was placed to cover the hybridization mixture and sealed with rubber cement. The probes and sample nucleic acids on the slide were co-denatured at 73° C. for 5 min and then hybridized at 37° C. for 16-24 hours on a ThermoBrite. After hybridization, the slide was soaked in 2×SSC and 0.1% NP-40 at room temperature until the coverslip came off. After the coverslip was removed, the slide was washed at 73° C. for 2 min in 0.4×SSC/0.3% NP-40, followed by 2×SSC/0.1% NP-40 for 1 min at room temperature. After washing, the slide was air-dried at room temperature, applied with 10 μl of nuclear counterstain DAPI I (1,000 ng/ml) and covered with a new 22×22 mm coverslip. The stained slide was ready for an evaluation under a fluorescent microscope or stored at −20° C. for future evaluation.

### Example 9

### Results of FISH Assays Using HPV16-SO Probe

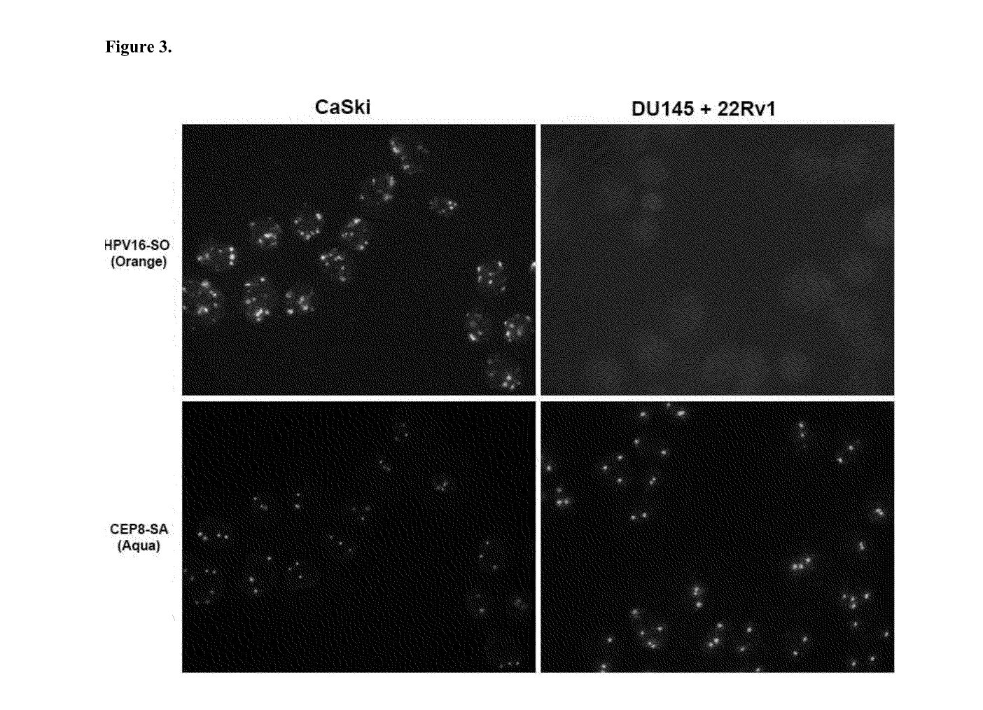

Results of HPV FISH assays using HPV16-SO probe in human cervical cancer cell line CaSki (positive control), and in a mixture of human prostate cancer cell lines DU145 and 22Rv1 (negative controls) as described in Example 6 are shown in FIG. 3. Inspection of DU145+22Rv1 slide revealed no cells with HPV16-SO probe hybridization (orange signal). As previously illustrated in FIG. 1 (A-B) and FIG. 2 (A), based on hybridization signals for the internal control CEP8-SA probe, DU145 cells harbored 3 copies of endogenous CEP8, while 22Rv1 cells contained just 2 copies. In contrast, when CaSki cells (which carry about 600 copies of integrated HPV-16 DNA per cell) were evaluated through an orange filter, a punctate staining pattern with multiple bright orange spots was observed in every single cell. These results demonstrate that the HPV16-SO probe is capable of directly and specifically detecting integrated HPV DNA in the nucleus. Of note, each CaSki cell appeared to have 2-3 copies of endogenous CEP8.

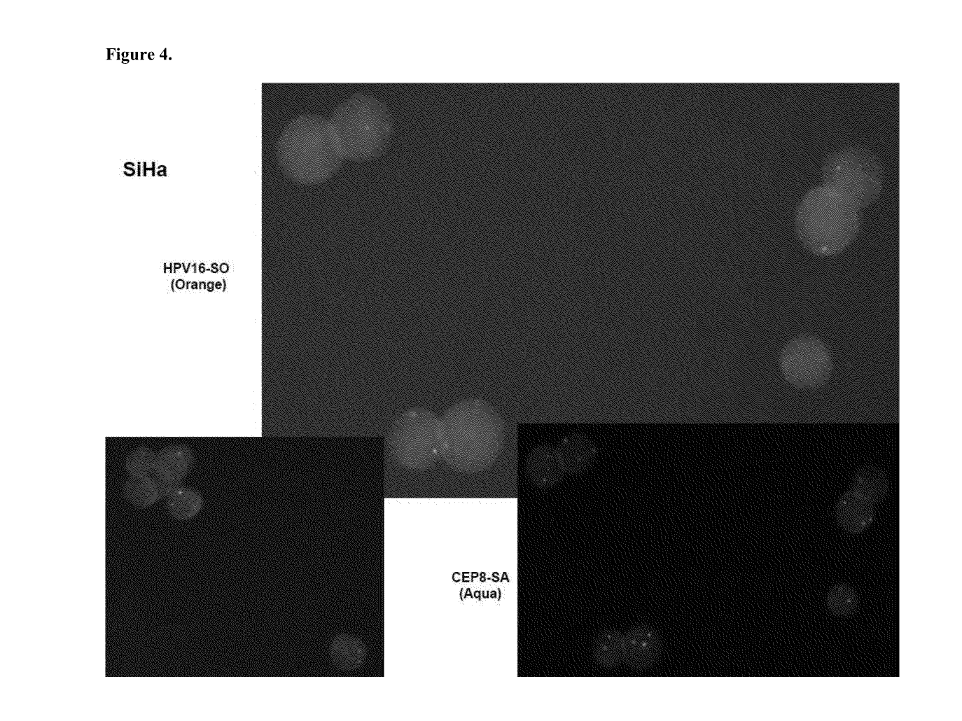

Results of an HPV FISH assay using HPV16-SO probe in human cervical cancer cell line SiHa as described in Example 7 are shown in FIG. 4. Contrary to CaSki cells, which carry about 600 copies of integrated HPV-16 DNA per cell and exhibit punctate staining patterns with multiple bright orange spots (FIG. 3), SiHa cells carry only 1 to 2 copies of integrated HPV-16 DNA per cell and exhibited 0, 1 or 2 orange spots in each cell (FIG. 4). Of 300 SiHa cells counted, 148 cells (49.3%) were found to carry either one or two orange spots. The results of CaSki, SiHa and DU145+22Rv1 together establish that the HPV16-SO probe specifically detects HPV DNA. Furthermore, the results of the SiHa cells demonstrate that this HPV16-SO probe is capable of detecting single copy of integrated HPV genome (˜8.0 kb) in the nucleus near 50% of the time. Similar to CaSki cells, each SiHa cell also appeared to have 2-3 copies of endogenous CEP8.

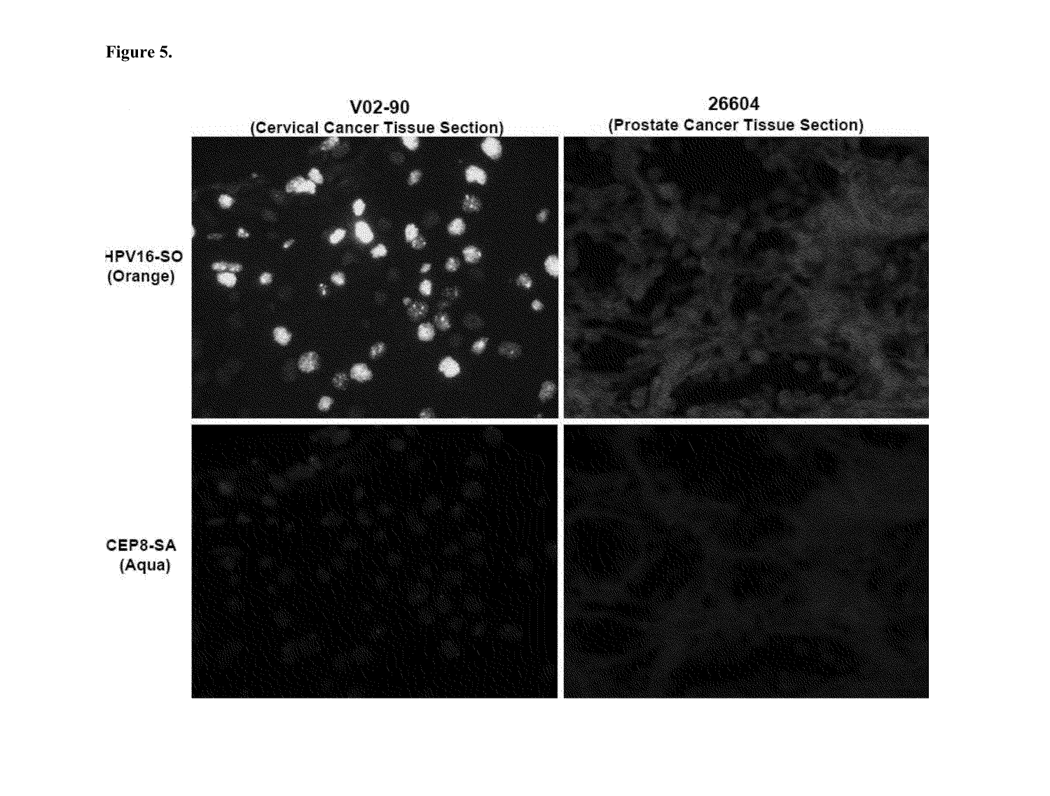

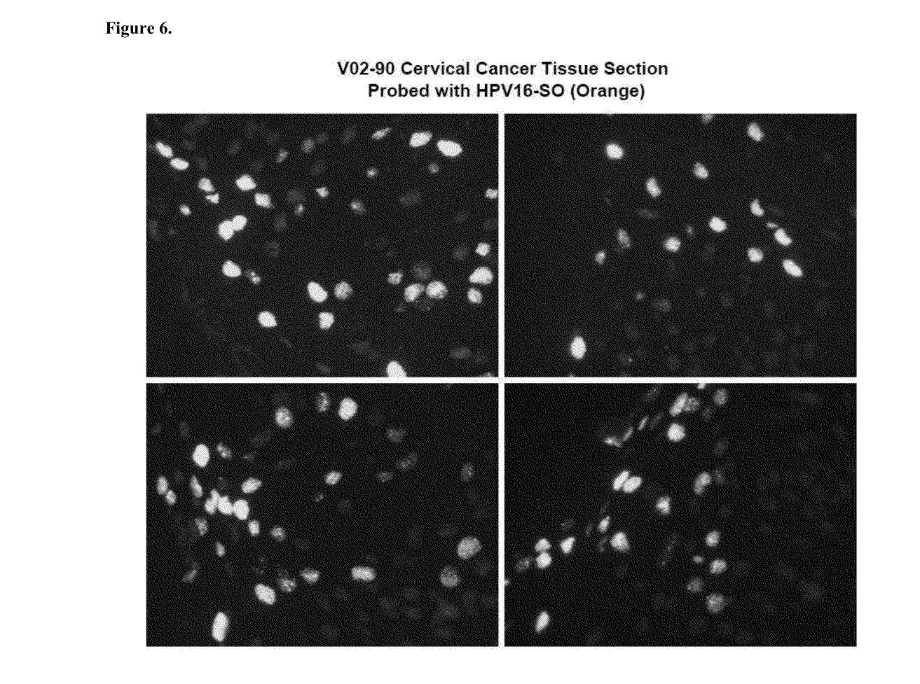

Results of HPV FISH assays using HPV16-SO probe in FFPE human cervical cancer tissue specimen (V02-90, positive control) and human prostate cancer tissue specimen (26604, negative control) as described in Example 8 are shown in FIG. 5. Similar to results obtained with CaSki cells (FIG. 3), hybridization with the HPV16-SO probe resulted in generating bright orange nuclear signal in a large number of epithelial cells of specimen V02-90. In addition, some cells, similar to SiHa cells (FIG. 4), exhibited just one or several spots of orange staining in the nuclei. In contrast, there was no evidence of HPV16-SO hybridization in the negative control 26604. Consistent with results obtained using tyramide-based signal amplification, Vysis Cervical FISH Probe Kit, http://www.abbottmolecular.com/products/oncology/fish/vysis-cervical-fish-probe-kit.html, the orange nuclear staining patterns in the HPV-infected cells of V02-90 were diffuse, punctate or mixed (see FIGS. 5 and 6). A diffuse staining pattern can be observed as complete orange staining of the nucleus and is indicative of an episomal HPV state. A punctate staining pattern can be observed as one or several individual spots of orange staining localized to the nucleus. The spots vary in size. A punctate staining pattern is indicative of an integrated HPV state. A mixed staining pattern contains both diffuse and punctate staining. Consistent with the results of the CaSki and SiHa cells illustrated in FIGS. 3 and 4, respectively, these results demonstrate that the HPV16-SO probe is capable of directly and specifically detecting the HPV-infected cells, including those carrying only a single copy of integrated HPV DNA and those in the FFPE tissue specimens.

In conclusion, the present study demonstrates the utility of a single directly-labeled SO probe (˜8 kb long) in the absence of signal amplification, to specifically detect both integrated proviral DNA as well as expressed viral RNA in XMRV-infected cells, and to specifically detect both integrated and episomal viral DNA in HPV-infected cells. In addition, these directly-labeled probes by themselves are capable of providing sufficient sensitivity to detect a single copy of integrated viral genome (˜8 kb long) as demonstrated by the detection of SiHa cells using the HPV16-SO probe.

### Example 10

### HPV FISH Assay in Cervical Cytology Specimens

This example employs a kit designed to identify HPV infected cells and determine copy number of the chromosomal regions 3q26 (TERC) and 8q24 (MYC) via fluorescence in situ hybridization (FISH) in cervical cytology specimens as an indicator of atypical squamous cells of undetermined significance (ASCUS), low-grade squamous intraepithelial lesions (LSIL), high-grade squamous intraepithelial lesions (HSIL) cases. The HPV staining pattern in infected cells can further indicate whether the virus is episomal or integrated into the host genome.

In the last decade, the etiological role of HPV infection in the development of cervical dysplasia and cancer has been well established. The use of HPV screening in conjunction with the Pap smear has increased the sensitivity of detecting dysplasia to nearly 100%. Despite the high frequency of HPV infection in dysplastic samples, only a fraction of HPV infected women will develop high-grade lesions and cervical cancer. Chromosomal instability at a numerical or structural level is a hallmark of malignant tumors. Frequent alterations of chromosome regions 3q26 (TERC)5-7 and 8q24 (MYC)8-10 in cervical malignant and pre-malignant lesions and cervical cancer cell lines have been demonstrated. The kit described in this example can be used as an aid in determining which samples have high risk HPV infection and harbor chromosomal aberrations associated with malignancy.

The kit described in this example includes the following three distinct FISH probes: (1) an HPV probe directly labeled with SpectrumOrange fluorophore TAMARA-SE (5-(and-6)-carboxytetramethylrhodamine, succinimidyl ester 5(6)-TAMRA, SE) as outlined in Example 1; (2) a TERC (3q26) probe labeled with SpectrumGold fluorescent label, which covers an approximately 495 kb region that contains the entire TERC gene; and (3) a MYC (8q24) probe labeled with SpectrumRed fluorescent label, which covers an approximately 821 kb region that contains the entire MYC gene.

The kit of the instant example finds use in assaying cervical specimens collected in PreservCyt fixative solutions, which can be deposited on slides using the ThinPrep instrument. Slides prepared in this fashion can be dried overnight at room temperature and then stored at −20° C. until hybridized. Hybridization is initiated by soaking the slides in 2×SSC, pH 7.0 at 73° C. for 2 min. Thereafter slides are incubated in pepsin solution (0.5 mg/ml in 10 mM HCl) at 37° C. for 10 min, followed by soaking the slides in 1×DPBS at room temperature for 5 min. After pepsin treatment the slides must be fixed. 50 ml of Formaldehyde Fixative solution is prepared by mixing 12.5 ml of 10% neutral-buffered formalin, 37 ml of 1×DPBS, and 0.5 ml of 2 M MgCl₂ and used to incubate the slides at room temperature for 5 min. After fixation, the slides are soaked in 1×DPBS at room temperature for 5 min. The slides can then be dehydrated in an ethanol series of 70%, 85%, and 100% ethanol for 1 min in each solution and air-dried.

Once the slides are prepared, the next steps involve probe preparation, hybridization and wash. Vortex and briefly spin the probe mixtures which are prepared as outlined in Example 1. Apply 10 μA of the probe mixtures on the target area of the slide; coverslip slides using 22×2 mm coverslips and seal with rubber cement. Co-denature the slides with probe mix at 73° C. for 3 min and then hybridize at 37° C. for 16-24 hours on a ThermoBrite or HYBrite. After hybridization, wash slides in 0.4×SSC/0.3% NP-40 for 2 min at 73° C., and then in 2×SSC/0.1% NP-40 for 1 min at room temperature. After washing, slides are air-dried at room temperature, applied with 10 μl of nuclear counterstain DAPI II (125 ng/ml) and covered with new coverslips. Stained slides are ready for evaluation under a fluorescent microscope.

In a cell infected with HPV, bright orange nuclear staining can be observed. Two categories of HPV staining patterns can be seen in clinical specimens: diffuse and punctate staining patterns. Diffuse staining pattern appears as complete orange staining of the nucleus. Diffuse staining pattern is indicative of an episomal HPV state. Punctate staining appears as one or several individual spots of orange staining localized to the nucleus. Spots can vary in size. Nuclear background can vary from completely dark to slightly orange. Punctate staining pattern is indicative of an integrated HPV state. True HPV staining should be localized to the nucleus as confirmed by DAPI and co-localization with the locus-specific probes TERC and MYC.

In a normal diploid cell, two gold signals should be observed for the TERC (3q26) probe and two red signals should be observed for the MYC (8q24) probe. Abnormal copy number of LSI TERC is indicated by more than two copies of the gold signal. Abnormal copy number of LSI MYC is indicated by more than two copies of the red signal. Cells that demonstrate both HPV positive staining and chromosomally abnormal copy number of at least one chromosomal locus (either TERC or MYC) are designated as “double-positive cells”.

In certain instances, when employing this kit for diagnosis, a sample will be considered positive for HPV infection if at least one HPV infected cell was found on a slide. In certain instances, when employing this kit for diagnosis, a sample will be considered positive for chromosome aneusomy if four or more HPV infected cells demonstrated copy number gain of at least one chromosome locus, either TERC or MYC (“double-positive cells”) on a slide.

### Example 11

### FISH Assays Using HPV16-SO Probe for Detection of HPV in FFPE Human Head and Neck Cancer Tissue Specimen

To test the capability of HPV16-SO in detecting the HPV-infected cells in human H&N cancer tissue, slides made from FFPE human head and neck cancer tissue sections were used. Histologically, the tissue represented Head and Neck Squamous Cell Carcinoma (HNSCC), and was procured from the Cooperative Human Tissue Network (CHTN), Midwestern Division, at the Ohio State University Medical Center, Columbus, Ohio. Two specimens were used in the experiment. The tissue IDs were 11-09-X022B and 11-09-X019B.

As a positive control, an FFPE cervical cancer specimen was utilized. This specimen, V02-90, was obtained from the University of Texas Southwestern Medical Center, Dallas, Tex., and was identified as HPV-positive using a biotin-labeled HPV probe set in combination of tyramide signal amplification (Vysis Cervical FISH Probe Kit, Abbott Molecular, Inc.). As a negative control, one FFPE human lung cancer tissue specimen was used, since lung cancer has not been shown to be associated with HPV infection. The lung cancer tissue was procured from the Cooperative Human Tissue Network (CHTN), Midwestern Division, at Ohio State University Medical Center. Both positive and negative control tissue sections were tested with HPV16-SO.

For the two head and neck cancer specimens, one lung cancer specimen, and one cervical cancer specimen, 5-micron sections were prepared and applied to microscope slides. The slides were baked at 56° C. for 4 hours and then stored at room temperature. A total of 4 slides (2 HNSCC, one cervical cancer and one lung cancer) were used in the experiment. Prior to hybridization, the slides were deparaffinated three times in Hemo-De solvent at room temperature for 5 min each, after which they were washed twice with 100% ethanol at room temperature for 1 min each. After washing, the slides were pretreated using Vysis Paraffin Pretreatment IV & Post-Hybridization Wash Buffer Kit (List No. 01N31-005): slides were incubated at 80° C. for 12 min in Vysis Pretreatment Solution (1N NaSCN), washed with water for 3 min at room temperature, digested with 1.5 mg/ml of pepsin in 0.1 N HCl at 37° C. for 20 min, and washed again with water for 3 min at room temperature. The slides then went through a series of three ethanol steps (70%, 85% and 100%) at room temperature for 1 min each. After dehydration, the slides were air-dried at room temperature and ready for hybridization with the HPV16-SO probe.

10 μl of the HPV16-SO probe mix was prepared by mixing 100 ng HPV16-SO probe, 100 ng CEP8-SA probe, 1000 ng sonicated human placental DNA, 250 ng human Cot-1 DNA, and 7 μl LSI/WCP hybridization buffer. 10 μl of the probe mixture was then applied on the target area of the slide. A 22×22 mm coverslip was placed to cover the hybridization mixture and sealed with rubber cement. The probes and sample nucleic acids on the slide were co-denatured at 73° C. for 5 min and then hybridized at 37° C. for 16-24 hours on a ThermoBrite. After hybridization, the slide was soaked in 2×SSC and 0.1% NP-40 at room temperature until the coverslip came off. After the coverslip was removed, the slide was washed at 73° C. for 2 min in 0.7×SSC/0.3% NP-40, followed by 2×SSC/0.1% NP-40 for 1 min at room temperature. After washing, the slide was air-dried at room temperature, applied with 10 μl of nuclear counterstain DAPI I (1,000 ng/ml) and covered with a new 22×22 mm coverslip. The stained slide was ready for an evaluation under a fluorescent microscope or stored at −20° C. for future evaluation.

### Example 12

### Results of FISH Assays on Head and Neck Cancer Tissue Specimens Using HPV16-SO Probe

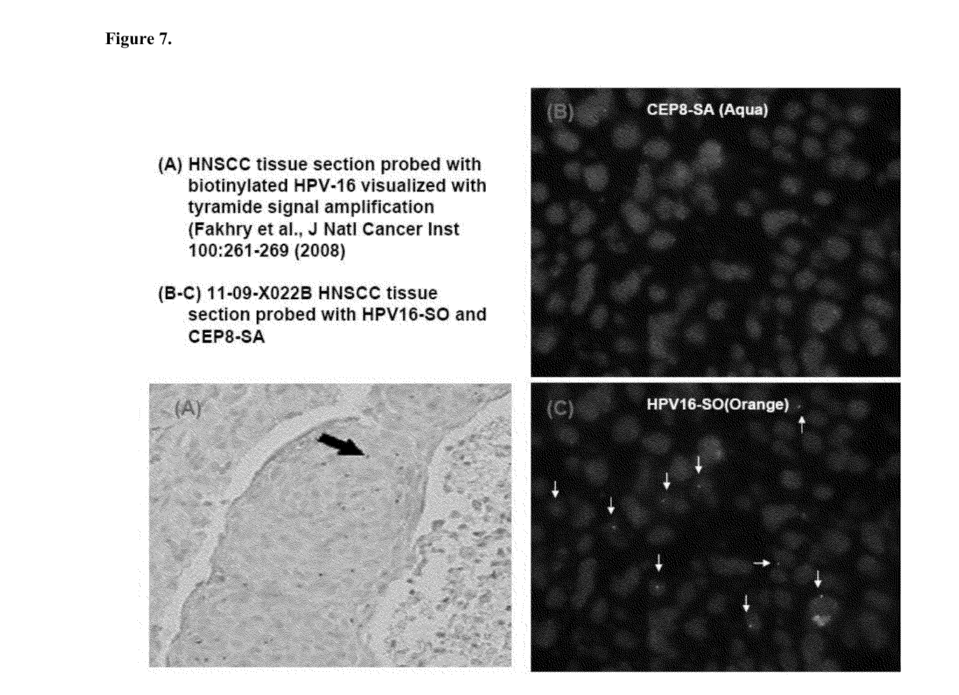

Results of HPV FISH assays on head and neck squamous cell carcinoma (HNSCC) tissue specimens using the HPV16-SO probe as described in Example 11 are shown in FIG. 7. Evaluated through an orange filter, one of the two HNSCC specimens, 11-09-X022B, was found to have HPV orange staining in a few particular squamous cell areas (FIG. 7, C). The observed HPV staining pattern was punctate and appeared very similar to an HPV-positive HNSCC sample with a score of “1+” (one focal hybridization signal in scattered tumor cell nuclei) (FIG. 7, A) reported by Fakhry et el. (J Natl Cancer Inst 100:261-269 (2008)). In the published study, a biotinylated HPV-16 probe and the tyramide signal amplification system with a chromogenic dye DAB (GenPoint; Dako, Carpinteria, Calif.) were used to detect HPV-16 DNA in specimens. The HPV16-SO probe, in this experiment, generated a large number of epithelial cells with HPV nuclear staining in the cervical cancer tissue specimen V02-90 (a positive control) as seen in FIGS. 5 and 6, but not in the lung cancer tissue specimen (a negative control) (data not shown). These results demonstrate that the HPV16-SO probe in the absence of signal amplification is capable of directly and specifically detecting the HPV-infected cells in HNSCC samples. Of note, specimen 11-09-X022B on average appeared to have 2 copies of endogenous CEP8 per cell (FIG. 7, B).

Following the above experiment, 5 more HNSCC specimens from CHTN were also tested for the presence of HPV-16 using the same HPV16-SO probe and protocol.

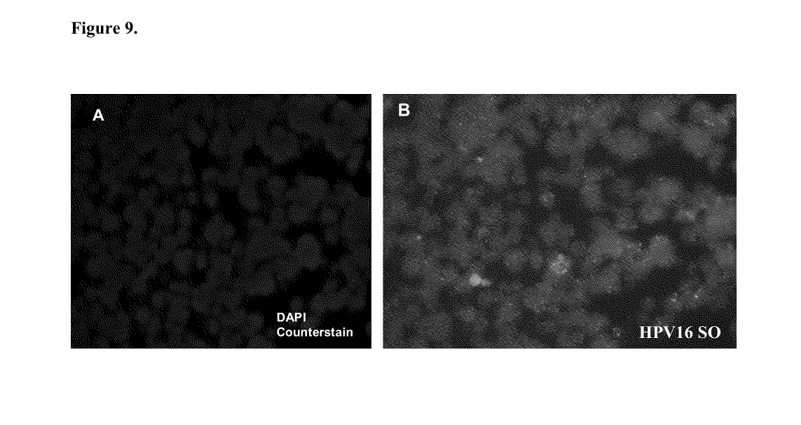

As shown in FIG. 9, one of the five HNSCC specimens tested, M1120510A(Q), was found positive for HPV-16 as proven by the appearance of an extensive orange nuclear staining with the HPV16-SO probe. The observed HPV staining pattern was punctate with two or more signals per nucleus in many cells and appeared very similar to an HPV-positive HNSCC sample with a score of “3+” reported by Fakhry et al., J Natl Cancer Inst 100:261-269 (2008).

Furthermore, the HPV-16 FISH results for three of the five HNSCC samples were compared with the results of p16 IHC and HPV in situ hybridization reported independently from CHTN as shown below. (See Table 2).

TABLE 2

| | | External Pathology Report | |
|---|---|---|---|
| | | Provided by CHTN | |
| | Abbott HPV16 | Chromogenic HPV | |
| Specimen ID | FISH Result | in situ Hybridization | p16 IHC |
| M1120602A | Negative | HPV16 Negative | Positive |
| M1120544A | Negative | HPV-Hi Negative | Positive |
| M1120510A | Positive | HPV-Hi Positive | Positive |

Singhi, et al. reported that on a direct comparison of p16 IHC and HPV-16 ISH with a discordancy rate of 7%. The discrepancies exclusively involved cancers that were negative for HPV-16 by ISH but p16 positive by IHC. The authors claimed that in one third of these discordant cases, high p16 expression was because of the presence of a non-16 HPV type, as confirmed by HPV ISH for additional oncogenic types. The remaining discordant cases may reflect the imperfections of p16 as a surrogate marker. By using E6/E7 mRNA levels as conclusive evidence of HPV involvement, others have shown that positive p16 immunostaining is 100% sensitive and 79% specific (A Singhi et al., Cancer 2010; 116:2166-73). In conclusion, our HPV FISH results with the HPV16-SO probe on the HNSCC specimens are in good agreement with those performed by CHTN, further demonstrating that our directly-labeled HPV16-SO probe is capable of directly and specifically detecting the HPV-infected cells in HNSCC samples.

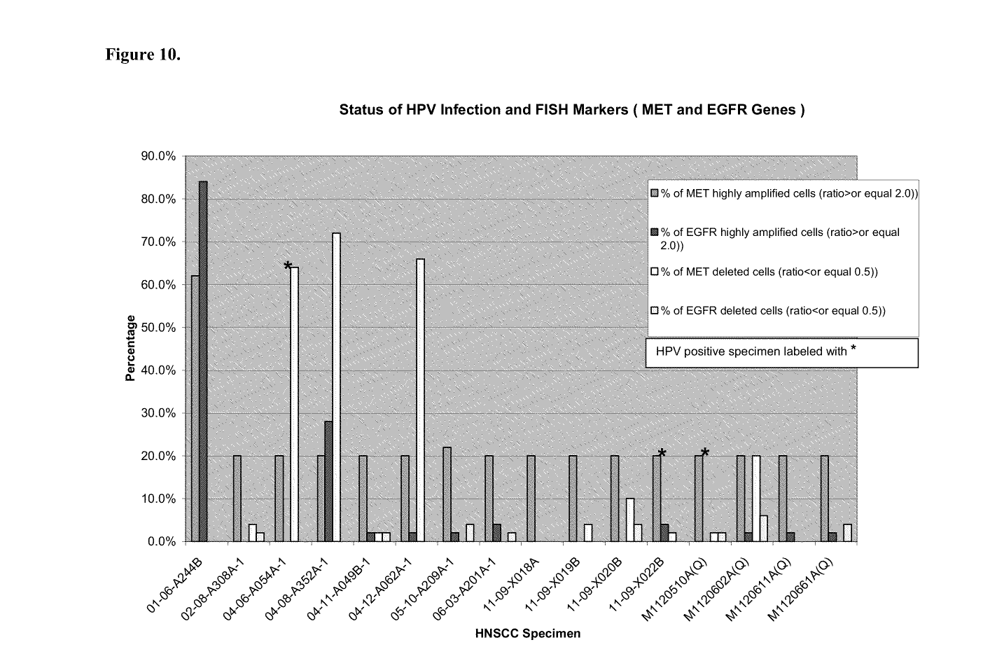

In summary, FIG. 10 illustrates the status of HPV16-SO staining and two chromosomal FISH probes (MET and EGFR) on sixteen HNSCC specimens as described above in the section of Combinations with Other FISH Markers, paragraphs [0063]-[0067], and shows the feasibility of combining the HPV16-SO probe with other chromosomal marker probes in FISH analysis.

### Example 13

### Results of FISH Assays on Head and Neck Cancer Tissue Specimens Using EGFR, MET, CEP7, and HPV16 Probes

Twenty-three Head and Neck Squamous Cell Carcinoma (HNSCC) specimens were analyzed using three FISH probes (MET, EGFR and CEP7). The hybridization followed the protocol described below.

First, the slides were deparaffinated three times in Hemo-De solvent at room temperature for 5 min each, after which they were washed twice with 100% ethanol at room temperature for 1 min each. After washing, the slides underwent pretreatment which included: incubation at 80° C. for 35 min in 1×SSC buffer, pH 7.0; followed by a water wash for 3 min at room temperature; digestion with 1.5 mg/ml of pepsin in 0.1 N HCl at 37° C. for 20 min; and a second water wash for 3 min at room temperature. The slides were then washed in ethanol in three steps (70%, 85% and 100%) at room temperature for 1 min each. After dehydration, the slides were air-dried at room temperature and ready for hybridization with the FISH probes (MET, EGFR and CEP7).

10 μl of the FISH probe set was prepared by mixing 300 ng EGFR-SGn probe, 250 ng MET-SR, 100 ng CEP7-SA probe, 2000 ng sonicated human placental DNA, 500 ng human Cot-1 DNA, and 7 μl LSI/WCP hybridization buffer. 10 μl of the probe mixture was then applied on the target area of the slide. A 22×22 mm coverslip was placed to cover the hybridization mixture and sealed with rubber cement. The probes and sample nucleic acids on the slide were co-denatured at 73° C. for 5 min and then hybridized at 37° C. for 16-24 hours on a ThermoBrite. After hybridization, the slide was soaked in 2×SSC and 0.1% NP-40 at room temperature until the coverslip came off. After the coverslip was removed, the slide was washed at 73° C. for 2 min in 2×SSC/0.3% NP-40, followed by 2×SSC/0.1% NP-40 for 1 min at room temperature. After washing, the slide was air-dried at room temperature, applied with 10 μl of nuclear counterstain DAPI I (1,000 ng/ml) and covered with a new 24×30 mm coverslip. The stained slide was ready for an evaluation under a fluorescent microscope or stored at −20° C. for future evaluation within 10 days after the hybridization.

Examination of the FFPE slides: sixteen HNSCC slides were enumerated successfully. Cell nuclei were visualized with a DAPI filter. Locating of the tumor epithelial cell areas as well as enumeration were performed under at 60× magnification with single bandpass filters for the respective fluorophores (red, green and aqua). The numbers of fluorescent signals from MET-SR (red), EGFR-SGn (green) and CEP7-SA (aqua) were recorded. A total of 50 abnormal FISH cells were identified that had at least one probe signal indicating target amplification or deletion.

Data analysis: Enumeration results were analyzed using JMP statistical software, version 9 (SAS Institute Inc., Cary, N.C.), and figures were drawn using Microsoft Excel 2003.

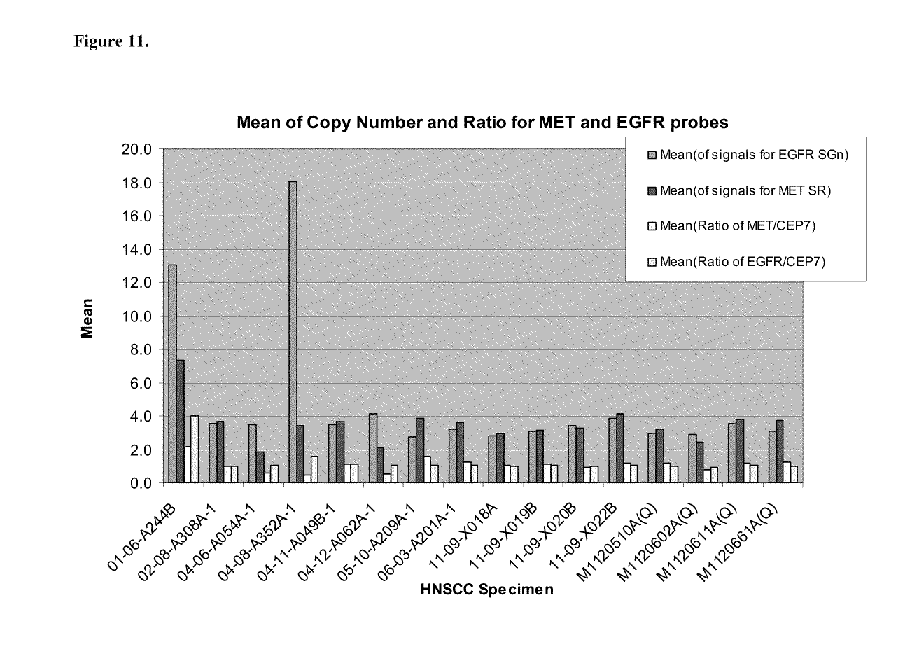

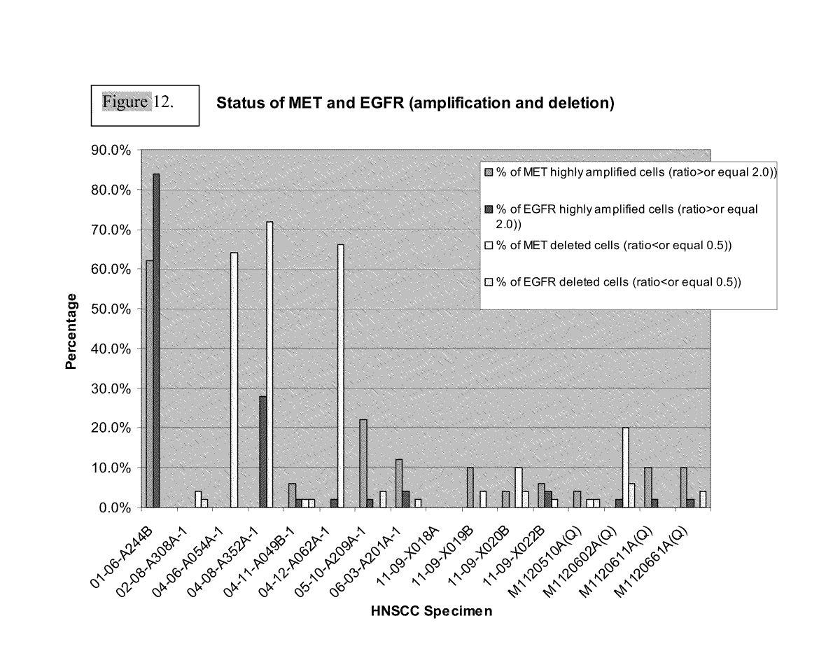

Results: Chromosomal patterns and the number of chromosomally abnormal cells were evaluated and recorded. A normal cell has a diploid genome, with 2 copies of each chromosome, and therefore 2 FISH signals for each locus assessed. An abnormal cell has either less than two (deletion) or greater than 2 (copy number gain) signals. HPV staining patterns and the scale of HPV positivity (1+, 2+, and 3+) were evaluated and recorded as well. Average copy number per cell for each specimen was calculated for chromosome markers MET and EGFR, respectively; as well as the average ratios of MET/CEP7 and EGFR/CEP7 in each specimen (n=16), as shown in FIG. 11. Additionally, the percentages of highly amplified and/or deleted cells were calculated for each of the two (MET and EGFR) and the ratio of MET/CEP7 and EGFR/CEP7, respectively. The definition of highly amplified cells refers to the ratio of MET/CEP7≧2 and/or EGFR/CEP7≧2, while the definition of MET and EGFR deleted cells refers to the ratio of MET/CEP7≦0.5 and ratio of EGFR/CEP7≦0.5, respectively. The results are summarized in FIG. 12. In summary, both the gain (amplification) and loss (deletion) of chromosome copy number were observed for the MET probe, while only amplification was observed for the EGFR probe.

The sixteen HNSCC specimens examined included the following tumor subtypes: oral cavity, larynx, oropharynx, tongue and tonsil. Under different tumor anatomic sites in HNSCC, the average percentages of highly amplified and/or deleted cells were calculated for both MET and EGFR chromosome markers, respectively, as shown in Table 3.

TABLE 3
Average percentages of cells with MET and EGFR
abnormalities in different tumor anatomic sites of HNSCC

| | Average percentage | | | |
|---|---|---|---|---|
| | Highly | Highly | MET | EGFR |
| | amplified | amplified | Deleted | Deleted |
| Tumor | MET cells | EGFR cells | cells | cells |
| Anatomic | (ratio of | (ratio of | (ratio of | (ratio of |
| Site in | MET/ | EGFR/ | MET/ | EGFR/ |
| HNSCC (n) | CEP7 ≧ 2) | CEP7 ≧ 2) | CEP7 ≦ 0.5) | CEP7 ≦ 0.5) |
| Oral Cavity | 7.0% | 1.7% | 2.7% | 1.0% |
| (n = 6) | | | | |
| Larnyx (n = 5) | 15.6% | 17.6% | 14.0% | 1.6% |
| Oropharynx | 0.0% | 15.0% | 69.0% | 0.0% |
| (n = 2) | | | | |
| Tongue (n = 2) | 2.0% | 1.0% | 11.0% | 4.0% |
| Tonsil (n = 1) | 22.0% | 2.0% | 0.0% | 4.0% |

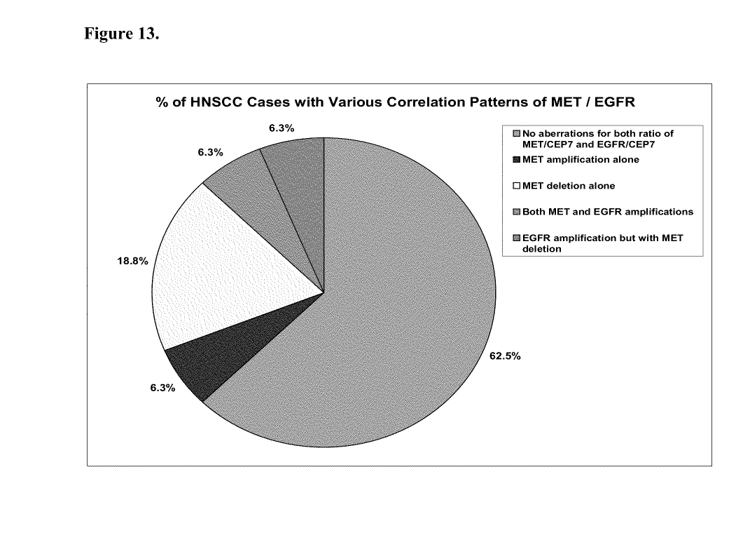

Furthermore, we have analyzed the relationship between the chromosome markers, MET and EGFR. The correlation for MET and EGFR gene was presented in various relationships (cutoff of >20% of cells with either amplification or deletion was used for both MET and EGFR), such as: 1.) MET and EGFR amplified simultaneously (6.3%); 2.) MET amplified (6.3%) or deleted (18.8%) alone; 3.) MET deleted while EGFR amplified (6.3%); and 4.) No aberrations for both ratio of MET/CEP7 and EGFR/CEP7 (62.5%). This correlation is shown in the FIG. 13.

Subsequently, the combination of HPV16 and FISH probe was tested further on fourteen of the sixteen HNSCC examined specimens in order to confirm the hybridization can be done in one hybridization reaction. The combined HPV/FISH probe set was a mixture of HPV16-SO with EGFR-SGn and CEP7-SA. The hybridization was performed with the following steps.

The slides were deparaffinated three times in Hemo-De solvent at room temperature for 5 min each, after which they were washed twice with 100% ethanol at room temperature for 1 min each. After washing, the slides underwent pretreatment. The slides were incubated at 80° C. for 35 min in 1×SSC buffer, pH 7.0, washed with water for 3 min at room temperature, digested with 1.5 mg/ml of pepsin in 0.1 N HCl at 37° C. for 20 min, and washed again with water for 3 min at room temperature. The slides then went through a series of three ethanol steps (70%, 85% and 100%) at room temperature for 1 min each. After dehydration, the slides were air-dried at room temperature and ready for hybridization with the HPV/FISH probe set (HPV16, EGFR and CEP7).

10 μl of the FISH probe set was prepared by mixing 100 ng HPV16-SO probe, 400 ng EGFR-SGn probe, 100 ng CEP7-SA probe, 1000 ng sonicated human placental DNA, 250 ng human Cot-1 DNA, and 7 μl LSI/WCP hybridization buffer. 10 μl of the probe mixture was then applied on the target area of the slide. A 22×22 mm coverslip was placed to cover the hybridization mixture and sealed with rubber cement. The probes and sample nucleic acids on the slide were co-denatured at 73° C. for 5 min and then hybridized at 37° C. for 16-24 hours on a ThermoBrite. After hybridization, the slide was soaked in 2×SSC and 0.1% NP-40 at room temperature until the coverslip came off. After the coverslip was removed, the slide was washed at 73° C. for 2 min in 0.4×SSC/0.3% NP-40, followed by 2×SSC/0.1% NP-40 for 1 min at room temperature. After washing, the slide was air-dried at room temperature, applied with 10 μl of nuclear counterstain DAPI I (1,000 ng/ml) and covered with a new 24×30 mm coverslip. The stained slide was ready for an evaluation under a fluorescent microscope or stored at −20° C. for future evaluation within 10 days after the hybridization.

In a cell infected with HPV, bright orange nuclear staining can be observed and illustrated as punctate HPV staining pattern. Punctate staining appears as one or several individual spots of orange staining localized to the nucleus. Spots can vary in size. Nuclear background can vary from completely dark to slightly orange. Punctate staining pattern is indicative of an integrated HPV state. True HPV staining should be localized to the nucleus as confirmed by DAPI and co-localization with the locus-specific probes EGFR (7p12) and centromere CEP7.

In a normal diploid cell, two green signals should be observed for the EGFR (7p12) probe and two aqua signals should be observed for the CEP7 probe. Abnormal copy number of LSI EGFR is indicated by more than two copies of the green signal. Abnormal copy number of CEP7 is indicated by more than two copies of the aqua signal.

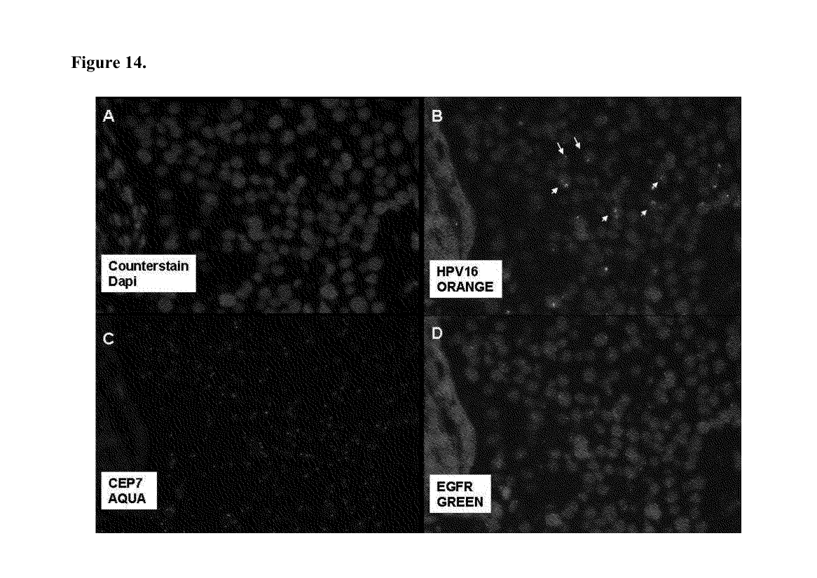

Images of one example (sample ID: 04-06-A054A-1) for this combined HPV/FISH probe set (HPV16-SO/EGFR-SGn/CEP7-SA) are shown in FIG. 14.

A pattern of punctate orange nuclear staining from the HPV16-SO probe was observed in certain areas of the specimen (FIG. 14, B). In addition of testing positive for HPV-16, this specimen on average appeared to have abnormal copy numbers (3 copies per cell) of both CEP7 and EGFR (FIG. 14, C and D, respectively). These results demonstrate that a multi-color probe set including HPV and chromosome probes can be used in FISH analysis.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. Furthermore, patents, patent applications, publications, procedures, and the like are cited throughout this application, the disclosures of those materials are hereby expressly incorporated herein by reference in their entireties.

What is claimed is:

1. A method of diagnosing a viral infection in a subject comprising:
(a) contacting a sample from said subject with a directly-labeled nucleic acid composition capable of hybridizing to a viral genomic sequence, wherein said viral genome is 5 kb-10 kb in length,
(b) washing said contacted sample to remove un-hybridized directly-labeled nucleic acid composition; and
(c) assaying said sample for the presence of the directly-labeled nucleic acid composition;
wherein the presence of the directly-labeled nucleic acid composition is indicative of a viral infection in said subject.

2. The method of claim 1 wherein the viral genomic sequence is selected from the group consisting of: XMRV, HTLV, HCV, HIV, and HPV viral genomic sequences.

3. The method of claim 1 wherein the sample is a cytological sample.

4. The method of claim 1 wherein the sample is a tissue sample.

5. The method of claim 4 wherein the tissue sample is a Formalin-Fixed Paraffin-Embedded tissue sample.

6. The method of claim 3 wherein the presence of said directly-labeled nucleic acid composition in a single cell of said sample is indicative of a viral infection in said subject.

7. The method of claim 3 wherein the presence of said directly-labeled nucleic acid composition in 1%-50% of the cells of said sample is indicative of a viral infection in said subject.

8. A method of diagnosing a predisposition to cancer in a subject comprising:
(a) contacting a sample from said subject with a directly-labeled nucleic acid composition capable of hybridizing to a viral genomic sequence, wherein said viral genome is 5 kb-10 kb in length,
(b) washing said contacted sample to remove un-hybridized directly-labeled nucleic acid composition; and
(c) assaying said sample for the presence of the directly-labeled nucleic acid composition;
wherein the presence of the directly-labeled nucleic acid composition is indicative of a predisposition to cancer in said subject.

9. The method of claim 8 wherein the viral genomic sequence is selected from the group consisting of: XMRV, HTLV, HCV, HIV, and HPV viral genomic sequences.

10. The method of claim 8 wherein the viral genomic sequence is HPV.

11. The method of claim 10 wherein the cancer is cancer of cervix, uterus, anus, oropharynx, penis, vagina, or vulva.

12. The method of claim 8 wherein the sample is a cytological sample.

13. The method of claim 8 wherein the sample is a tissue sample.

14. The method of claim 13 wherein the tissue sample is a Formalin-Fixed Paraffin-Embedded tissue sample.

15. The method of claim 12 wherein the presence of said directly-labeled nucleic acid composition in a single cell of said sample is indicative of a predisposition to cancer in said subject.

16. The method of claim 12 wherein the presence of said directly-labeled nucleic acid composition in 1%-50% of the cells of said sample is indicative of a predisposition to cancer in said subject.

17. A method of discriminating between cells expressing viral RNA and cells comprising episomal viral DNA, comprising:
(a) contacting a first sample from said subject with a directly-labeled nucleic acid composition capable of hybridizing to a viral genomic sequence, wherein said viral genome is 5 kb-10 kb in length,
(b) washing said contacted sample to remove un-hybridized directly-labeled nucleic acid composition; and
(c) assaying said sample for a signal indicative of the presence of the directly-labeled nucleic acid composition;
(d) contacting a second sample from said subject with a directly-labeled nucleic acid composition capable of hybridizing to a viral genomic sequence, wherein said viral genome is 5 kb-10 kb in length and said second sample has been pre-treated with RNase;
(e) washing said contacted sample to remove un-hybridized directly-labeled nucleic acid composition; and
(f) assaying said sample for the presence of a signal indicative of the directly-labeled nucleic acid composition; and
(g) comparing the signal obtained in step (c) with the signal obtained in (f);
wherein when a diffuse staining pattern of the directly-labeled nucleic acid composition in step (c) is contrasted with a punctate staining pattern of the directly-labeled nucleic acid composition in step (f), such a contrast is indicative of virus integrated into the host genome that is actively expressing viral RNA; and wherein the presence of a diffuse staining pattern in both (c) and (f) is indicative of the presence of episomal viral DNA.

18. A method of diagnosing a predisposition to cancer in a subject comprising:
(a) contacting a sample from said subject with a directly-labeled nucleic acid composition capable of hybridizing to a viral genomic sequence, wherein said viral genome is 5 kb-10 kb in length,
(b) washing said contacted sample to remove un-hybridized directly-labeled nucleic acid composition;
(c) simultaneously or sequentially contacting the sample with a nucleic acid composition capable of hybridizing to a chromosomal marker; and
(d) assaying said sample for the presence of the directly-labeled nucleic acid composition and the nucleic acid composition capable of hybridizing to a chromosomal marker;
wherein the presence of the directly-labeled nucleic acid composition and an abnormal signal from the nucleic acid composition capable of hybridizing to a chromosomal marker is indicative of a predisposition to cancer in said subject.

19. The method of claim 18 wherein the viral genomic sequence is selected from the group consisting of: XMRV, HTLV, HCV, HIV, and HPV viral genomic sequences.

20. The method of claim 18 wherein the chromosomal marker is selected from the group consisting of: EGFR (7p11.2), p53 (17p13.1), p16/CDKN2A (9p21), CCND1 (11q13.3), RB1 (13q14.2), TERT (5p15.33), MET (7q31.2), PIK3CA (3q26.32), TGFβ1 (19q13.2), MYC (8q24.21), PTEN (10q23.31), TERC (3q26.2) and DCC (18q21.2).

21. The method of claim 19 wherein the viral genomic sequence is HPV.

22. The method of claim 21 wherein the cancer is cancer of cervix, uterus, anus, oropharynx, penis, vagina, or vulva.

23. The method of claim 18 wherein the sample is a cytological sample.

24. The method of claim 18 wherein the sample is a tissue sample.

25. The method of claim 24 wherein the tissue sample is a Formalin-Fixed Paraffin-Embedded tissue sample.
